(19) United States
(12) Patent Application Publication
(10) Pub. No.: US 2019/0002387 A1
(43) Pub. Date: Jan. 3, 2019

(54) CYCLOBUTANE-1, 3-DIACID BUILDING BLOCKS

(71) Applicant: The University of North Dakota, Grand Forks, ND (US)

(72) Inventors: Qianli Chu, Grand Forks, ND (US); Zhihan Wang, Grand Forks, ND (US)

(21) Appl. No.: 16/024,817

(22) Filed: Jun. 30, 2018

Related U.S. Application Data
(60) Provisional application No. 62/527,590, filed on Jun. 30, 2017.

(51) Int. Cl. C07C 51/373; C08G 63/78; C07D 307/54

(52) U.S. Cl. CPC C07C 51/373; C07C 2601/04; C07D 307/54; C08G 63/78

(57) ABSTRACT

A method of making polymers utilizes cyclobutane-1,3-diacid (CBDA) molecules as polymer building blocks includes and linker molecules with a non-reactive R group and at least two reactive X groups used to create chemically stable polymers of CBDA. The resulting polymers are thermally, photochemically, and chemically stable.

### CROSS-REFERENCE TO RELATED APPLICATION(S)

This application claims the benefit of U.S. Provisional Application No. 62/527,590 filed Jun. 30, 2017 for “CYCLOBUTANE-1, 3-DIACID BUILDING BLOCKS” by Q, Chu and Z. Wang.

### STATEMENT OF GOVERNMENT INTEREST

This invention was made with government support under Grant number IIA-1355466 awarded by National Science Foundation. The government has certain rights in the invention.

### BACKGROUND

This application relates generally to polymer building blocks and specifically to cyclobutane-1,3-diacid polymer building blocks.

Synthetic polymers have a broad array of applications in industry. For instance, polyesters play an important role in clothing, food packaging, 3-D printing, construction, transportation, and biocompatible plastics. Building blocks for synthetic polymers must be stable molecules capable of producing polymer formations, and preferably should be chemically stable so that the resulting polymers can be used in a broad array of applications.

Diacids are widely used in modern materials. An example is the aliphatic diacid, adipic acid, used to make Nylon 66. Aromatic diacids have also found a variety of applications in materials. For instance, terephthalic acid, or benzene-1,4-dicarboxylic acid, is produced by chemical synthesis of crude oil. It is a building block in polyethylene terephthalate (PET), which is widely known for its use in plastic beverage bottles. Researchers are currently trying to find a biomass-based diacid to serve as an alternative to terephthalic acid. A prime candidate has been the furan-based building block 2,5-furandicarboxylic acid, which was named one of the top-12 value-added chemicals for “green” chemistry.

Despite the prevalence of the cyclobutane unit in many natural products and synthetic drugs, it is rarely seen in materials with industrial applications, most likely because concern about its stability has discouraged experimentation with this promising building block. When compared to five- and six-membered carbon rings, four-membered carbon rings are indeed less stable.

### SUMMARY

In one embodiment, a method of making a polyester includes providing a plurality of cyclobutane-1,3-diacid molecules, and polymerizing the plurality of cyclobutane-1,3-diacid molecules together with a plurality of linker molecules to create a polymer. Each of the plurality of linker molecules includes an R group and at least two X groups. The R group does not react with the cyclobutane-1,3-diacid molecules. The X groups do react with the cyclobbutane-1,3-diacid molecules.

In a second embodiment, a polymer includes a plurality of cyclobutane-1,3-diacid monomers, and a plurality of linking groups. Each of the plurality of cyclobutane-1,3-diacid monomers contains two carboxylic acid groups. Each of the plurality of linking groups includes an R group and at least two X groups. Each of the X groups is connected to one of the carboxylic acid groups in the plurality of cyclobutane-1,3-diacid monomers such that each of the plurality of linking groups chemically bonds to at least two of the plurality of cyclobutane-1,3-diacid monomers.

### BRIEF DESCRIPTION OF THE DRAWINGS

FIG. 1 depicts diacid building blocks for materials synthesis.

FIG. 2 depicts a method of using (1a,2a,3β,4β)-2,4-diphenylcyclobutane-1,3-dicarboxylic acid (CBDA-1) as a building block for polymer synthesis.

FIGS. 3A-3B show thermogravimetric analysis (TGA) and differential scanning calorimetry analysis (DSC) of CBDA-1 building blocks.

FIGS. 4A-4C picture the structure of CBDA-1 building blocks.

FIG. 5 shows synthesis of CBDA-1 and poly-α-truxillate.

FIG. 6 depicts a repeating unit of poly(ethylene-α-truxillate) (PEAT).

FIG. 7 shows thermostability of CBDA-1 polymer poly-α-truxillate.

FIG. 8 shows Oak Ridge Thermal Ellipsoid Plot (ORTEP) representation of CBDA-1 at 50% electron density.

FIG. 9 shows chemical structures of CBDA-1 (truxillic acid) stereoisomers.

FIG. 10 shows UV-Vis spectra of trans-cinnamic acid and CBDA-1 in ethanol.

FIGS. 11A-11G show NMR spectra of α-truxillic acid (CBDA-1).

FIGS. 12A-12D show NMR spectra of α-truxillate-dibutylaminium.

FIGS. 13A-13D show NMR spectra of PEAT.

FIGS. 14A-14D show NMR spectra of poly(1,4-butylene-α-truxillate) (PBAT).

FIGS. 15A-15D show NMR spectra of poly(1,5-pentylene-α- truxillate) (PPAT).

FIGS. 16A-16D show NMR spectra of poly(1,6-hexylene-α-truxillate) (PHAT).

FIG. 17 shows an FT-IR spectra of CBDA-1.

FIG. 18 shows an FT-IR spectra of PEAT.

FIG. 19 shows an FT-IR spectra of PBAT.

FIG. 20 shows an FT-IR spectra of PPAT.

FIG. 21 shows an FT-IR spectra of PHAT.

FIG. 22 shows powder X-ray diffraction of PEAT.

FIG. 23 shows powder X-ray diffraction of PBAT.

FIG. 24 shows powder X-ray diffraction of PPAT.

FIG. 25 shows powder X-ray diffraction of PHAT.

FIGS. 26A-26C show MS spectra of PEAT.

FIGS. 27A-27C show MS spectra of PBAT.

FIGS. 28A-28C show MS spectra of PPAT.

FIGS. 29A-29C show MS spectra of PHAT.

FIG. 30 shows DSC results for PEAT,

FIG. 31 shows DSC results for PBAT.

FIG. 32 shows DSC results for PPAT.

FIG. 33 shows DSC results for PHAT.

FIG. 34 shows TGA of CBDA-1 and poly-α-truxillate.

FIG. 35 is a table showing crystal data for cinnamic acid, CBDA-1 and its dibutylamine salt.

FIG. 36 depicts renewal diacid polymer building blocks from furfural.

FIG. 37 depicts a method of using (1α,2α,3β,4β)-2,4-di(furan-2-yl)cyclobutane-1,3-dicarboxylic acid (CBDA-5) or (1α,2α,3β,4β)-2,4-di(furan-2-yl)cyclobutane-1,3-diester (CBDE-1) as a building block for polymer synthesis.

FIGS. 38A-38B show thermogravimetric analysis (TGA) and differential scanning calorimetry analysis (DSC) of CBDA-5 and CBDE-1 building blocks.

FIGS. 39A-39E picture the structure of CBDA-5 building blocks.

FIG. 40 shows synthesis of CBDA-5 and CBDE-1 from furfural.

FIGS. 41A-41C show chemical structure and proton NMR of intermediate Compound 3 and CBDE-1, and X-ray diffraction analysis for CBDE-1.

FIGS. 42A-42B show proposed chemical structures of polyethylene cyclobutane-1,3-dicarboxylate (PEC-1).

FIGS. 43A-43C show NMR spectra of Compound 3.

FIGS. 44A-44F show NMR spectra of CBDE-1.

FIGS. 45A-45F show NMR spectra of CBDA-5.

FIG. 46 shows an FT-IR spectrum for Compound 3.

FIG. 47 shows an FT-IR spectrum for CBDE-1.

FIG. 48 shows an FT-IR spectrum for CBDA-5.

FIG. 49 shows an FT-IR spectrum for PEC-1.

FIG. 50 shows a UV-Vis spectrum of Compound 3, CBDE-1, and CBDA-5.

FIG. 51 shows an MS spectrum of CBDE-1.

FIG. 52 shows an MS spectrum of CBDA-5.

FIG. 53 shows an MS spectrum of PEC-1.

FIG. 54 shows TGA and DTG data for CBDE-1 and CBDA-5.

FIG. 55A shows TGA and DTG data for PEC-1.

FIG. 55B shows DSC data for PEC-1.

FIG. 56 shows crystal data of CBDE-1.

FIG. 57 shows crystal data of CBDA-5.

FIG. 58 is a table depicting crystal data of CBDE-1 and CBDA-5.

### DETAILED DESCRIPTION

Disclosed is a method of making polymers utilizing cyclobutane-1,3-diacid (CBDA) molecules as polymer building blocks as shown in FIG. 2. In this method, described in detail below, a linker molecule with reactive X groups and a non-reactive R group is used to create chemically stable polyesters.

Two different types of CBDA molecules are contemplated as building blocks: (1α,2α,3β,4β)-2,4-diphenylcyclobutane-1,3-dicarboxylic acid (CBDA-1) and (1α,2α,3β,4β)-2,4-di(furan-2-yl)cyclobutane-1,3-dicarboxylic acid (CBDA-5). CBDA-1 can produce variants of poly-α-truxillate, while CBDA-5 can produce polyethylene cyclobutane-1,3-dicarboxylate (PEC-1) or other varying polyesters. Depending on the attached functional groups, CBDA building block can produce polyesters with a variety of structures and properties.

CBDA can be used in material synthesis due to its great potential. CBDA shows outstanding thermal and sunlight stability. While its two carboxylic acid groups can be readily utilized in connecting with other molecules to form new materials, the cyclobutane ring is able to tolerate acids and bases, exhibiting good chemical stability. Additionally, CBDA can be included as a semi-rigid building block to synthesize polymers and other materials such as metal-organic frameworks (MOFs) and oligomers.

CBDA has sufficient thermal and photochemical/sunlight stability for many potential applications in materials. Incorporating CBDA into polymers requires a linker molecule capable of reacting with carboxylic acid. A series of aliphatic diols can be used as linker molecules, combining with CBDA-1 to create polyesters that showed excellent thermal and chemical stability.

### I. Polymer Synthesis From CBDA-1

A first variant of CBDA, (1α,2α,3β,4β)-2,4-diphenylcyclobutane-1,3-dicarboxylic acid (CBDA-1), which is also known as a-truxillic acid, can be used to produce polyesters. Compared to the other classic diacid building blocks, CBDA-1 represents a unique semi-rigid building block in material synthesis due to the presence of the small aliphatic ring.

Polymerization of CBDA-1 can produce poly-α-truxillate, including poly(ethylene-α-truxillate) (PEAT), poly(propylene-α-truxillate) (PPAT3), poly(1,4-butylene-α-truxillate) (PBAT), poly(1,5-pentylene-α-truxillate) (PPAT), and poly(1,6-hexylene-α-truxillate) (PHAT). A series of CBDA derived polyesters, including poly-α-truxillates, exhibit excellent stability.

### Overview of CBDA-1 Figures

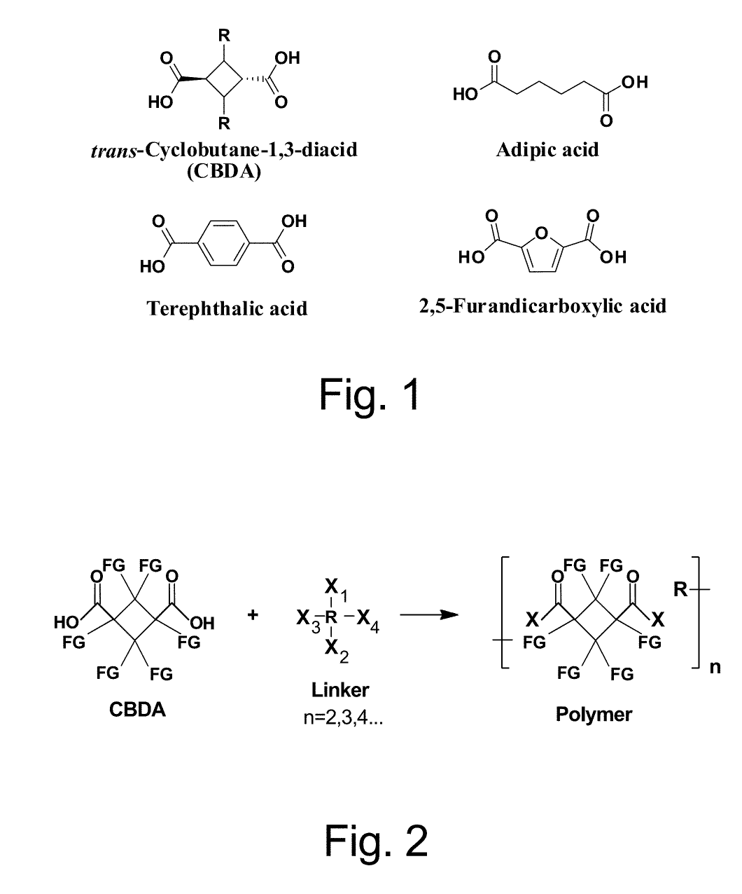

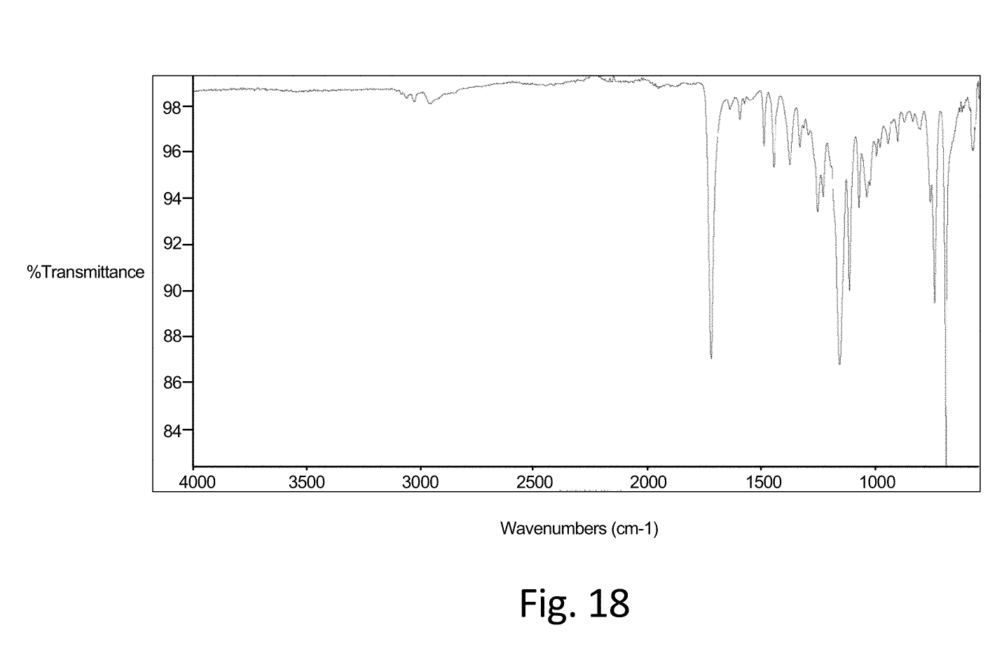

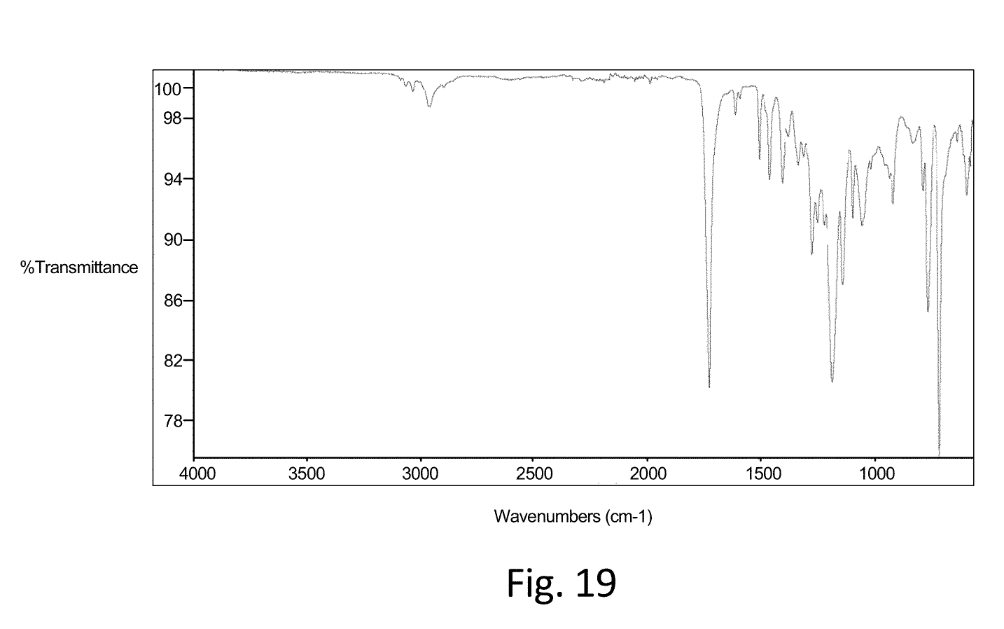

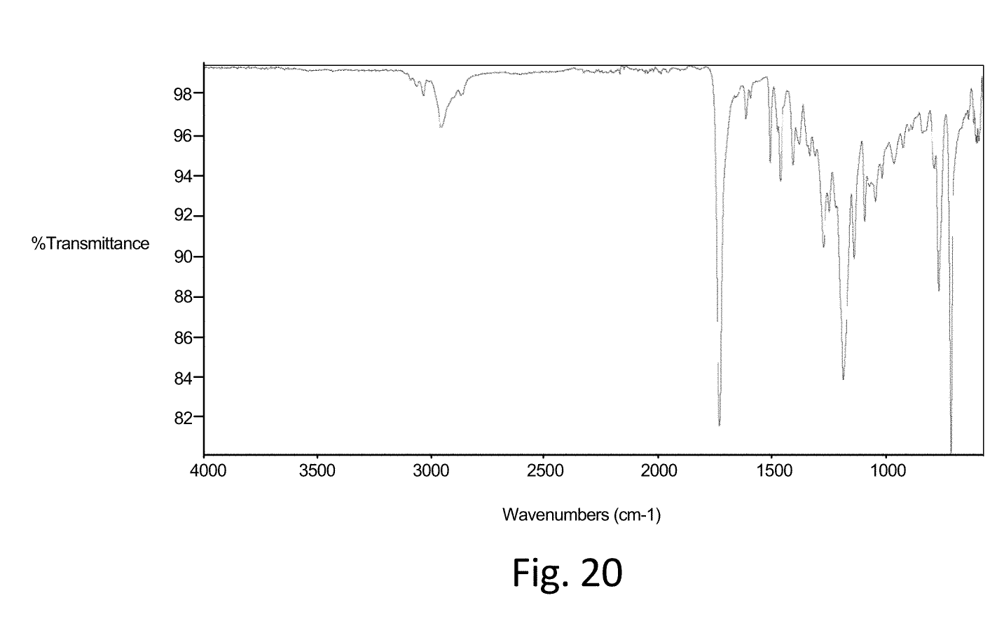

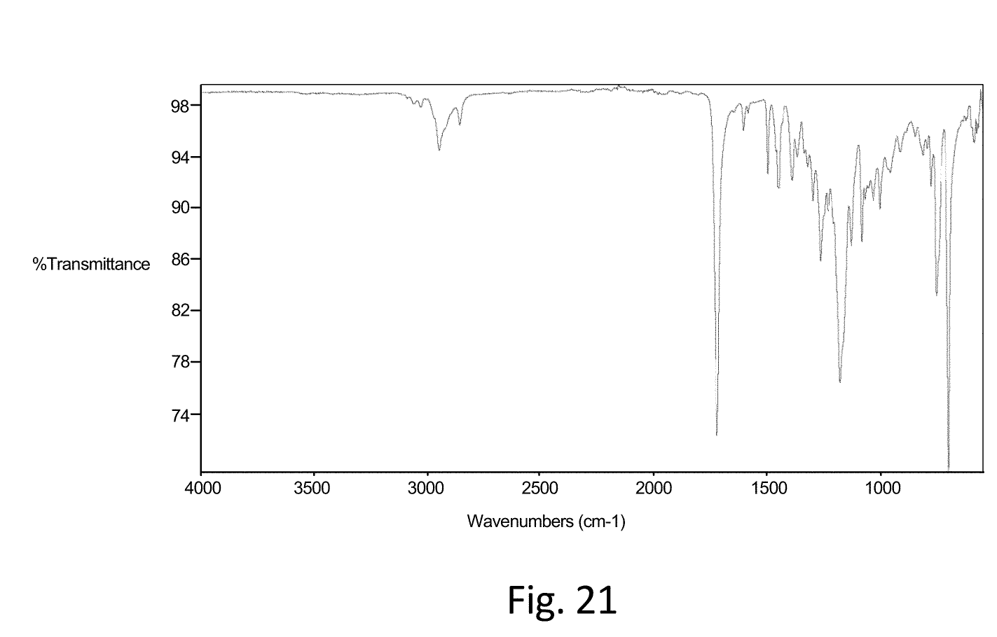

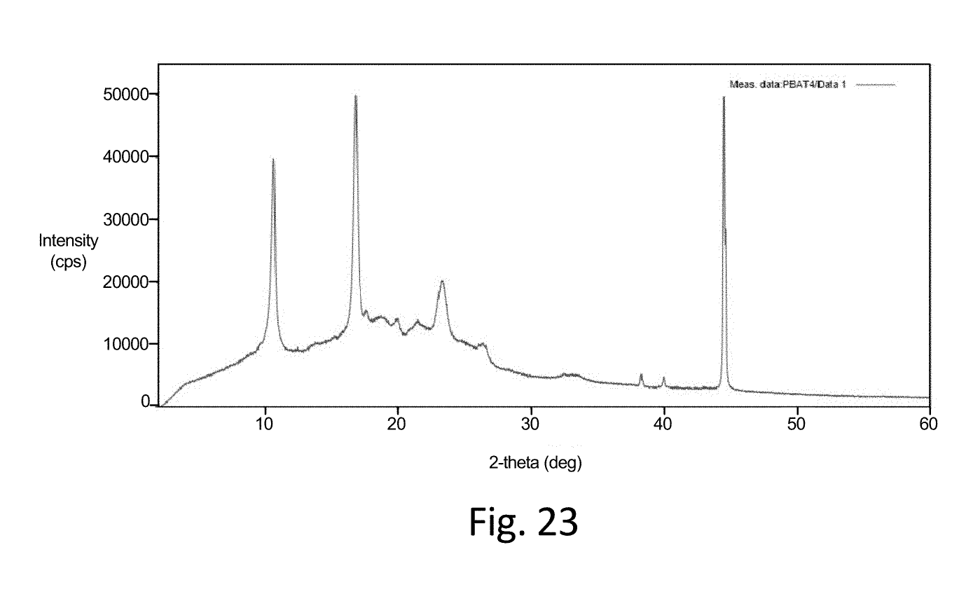

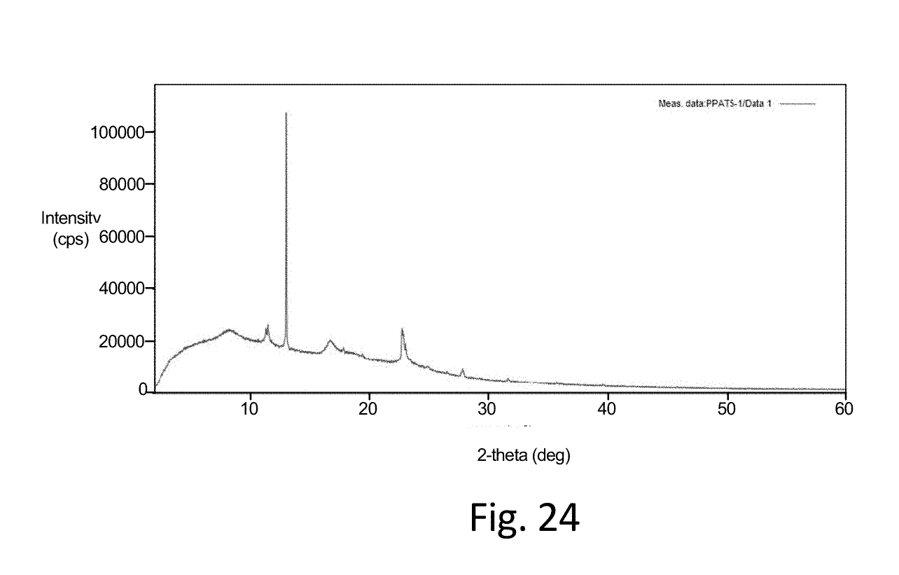

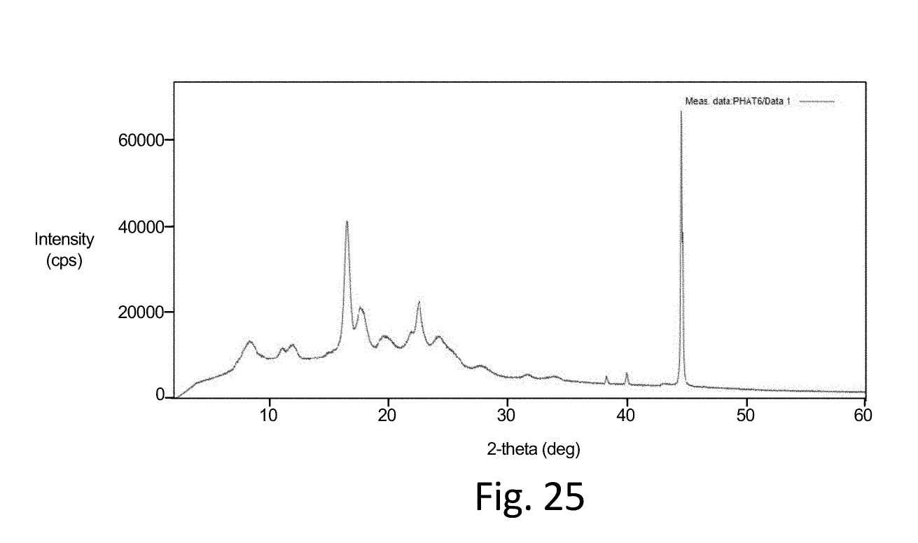

FIGS. 1-35 show laboratory results from production of CBDA-1 and CBDA-1 based polyesters. Various types of chemical analysis, including NMR, FT-IR, and MS spectra, TGA and DSC thermal analysis, and others were used to confirm that (1) CBDA-1 was produced, and the structure of CBDA-1 was as expected, and (2) polyesters from CBDA-1 were produced and their structure. Overall, CBDA-1 is a useful, semi-rigid and thermally chemically stable monomer. For these reasons, CBDA-1 is a suitable building block for creating polyesters.

FIGS. 1-2 illustrate chemical structures and reactions related to using CBDA as a building block of polymer and poly-esters. FIG. 1 depicts chemical structures of typical diacids, including a CBDA molecule, trans-Cyclobutane-1,3-diacid. FIG. 2 shows the reaction of a plurality of CBDA molecules with a plurality of linker molecules to create a CBDA polymer. The linker molecules each contain an R group that does not react with the carboxylic acid of the CBDA-1 molecules, and X groups which do react with the carboxylic acid of the CBDA-1 molecules.

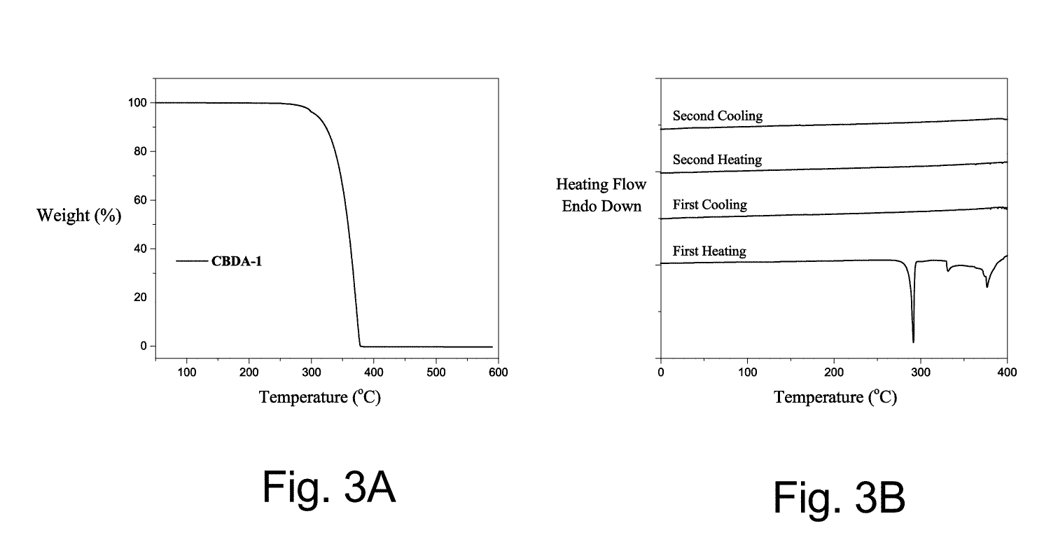

FIG. 3 shows analysis of CBDA-1 molecules. FIG. 3A shows a thermogravimetric analysis (TGA) of CBDA-1 that was recorded from 50° C. to 600° C. with a heating rate of 20° C. per minute under a nitrogen atmosphere. FIG. 3B shows differential scanning calorimetry (DSC) analysis of CBDA-1 molecules. DSC was recorded from 0° C. to 400° C. with a heating rate of 10° C. per minute under a nitrogen atmosphere. TGA and DSC analysis reveals that CBDA-1 is thermally stable enough to be used as a polymer building block.

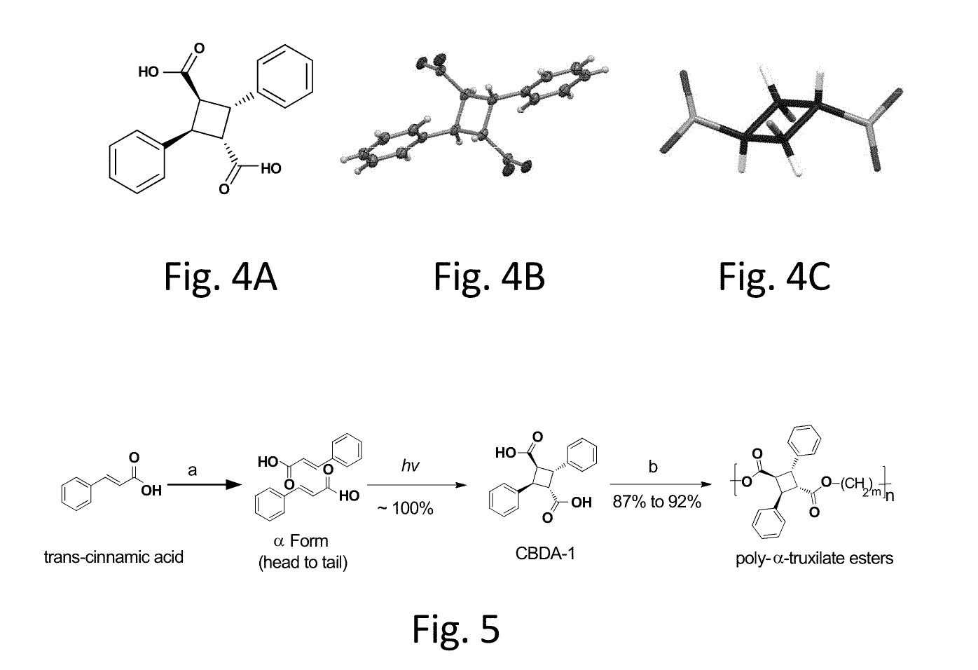

FIGS. 4A-4C show the structures of a CBDA-1 molecule. FIG. 4A shows the chemical structure of CBDA-1. FIG. 4B shows X-ray single crystal structure in an ORTEP representing 50% electron density of the monomer structure. FIG. 4C shows a single crystal structure in stick style with the cyclobutane ring highlighted and the two phenyl groups omitted for clarity.

FIG. 5 shows a scheme of synthesizing CBDA-1 molecules and poly-α-truxillate. To start, a commercially available trans-cinnamic acid is in α-form (that is, head-to-tail packed) and treated to form CBDA-1 molecules as described in detail above. Further crystallization of the trans-cinnamic acid is not necessary. CBDA-1 molecules are linked to form poly-α-truxillate as described in detail above.

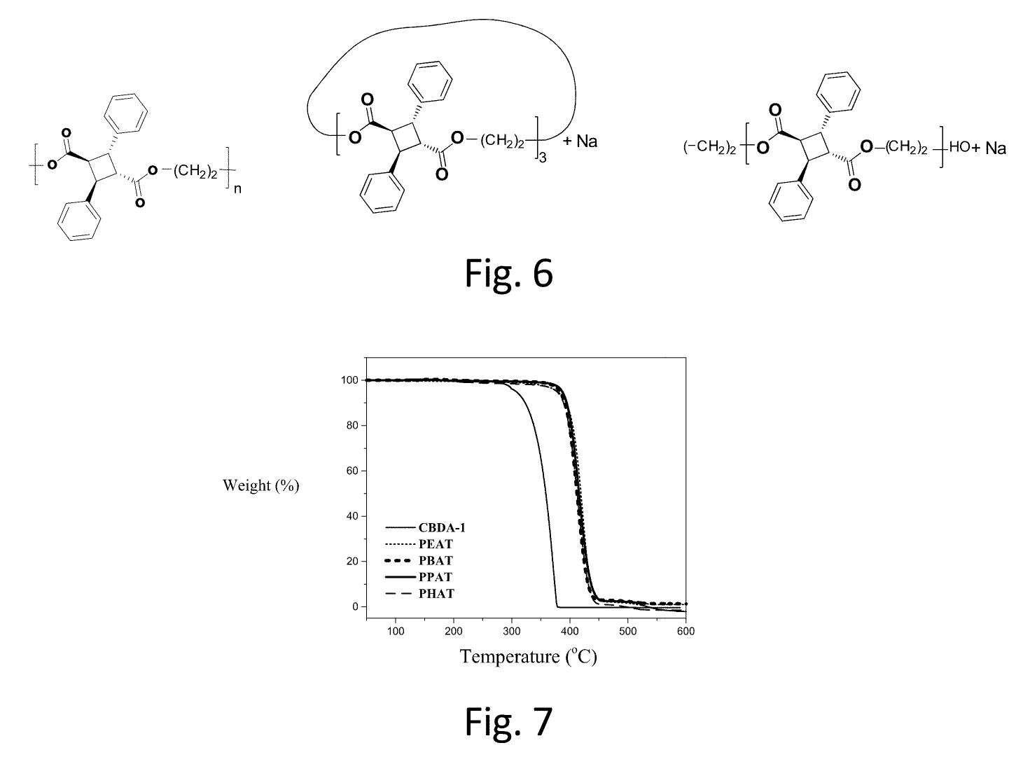

FIG. 6 shows a repeating unit of a poly-α-truxillate, specifically poly(ethylene-α-truxillate) (“PEAT”), in various polymers. PEAT was created from CBDA-1 molecules. FIG. 7 shows TGA testing with various poly-α-truxillates, including poly(ethylene-α-truxillate) (PEAT), poly(propylene-α-truxillate) (PPAT3), poly(1,4-butylene-α-truxillate) (PBAT), poly(1,5-pentylene-α-truxillate) (PPAT), and poly(1,6-hexylene-α-truxillate) (PHAT). The CBDA-1 molecule TGA curve is shown for reference. All polymers were thermally stable when tested.

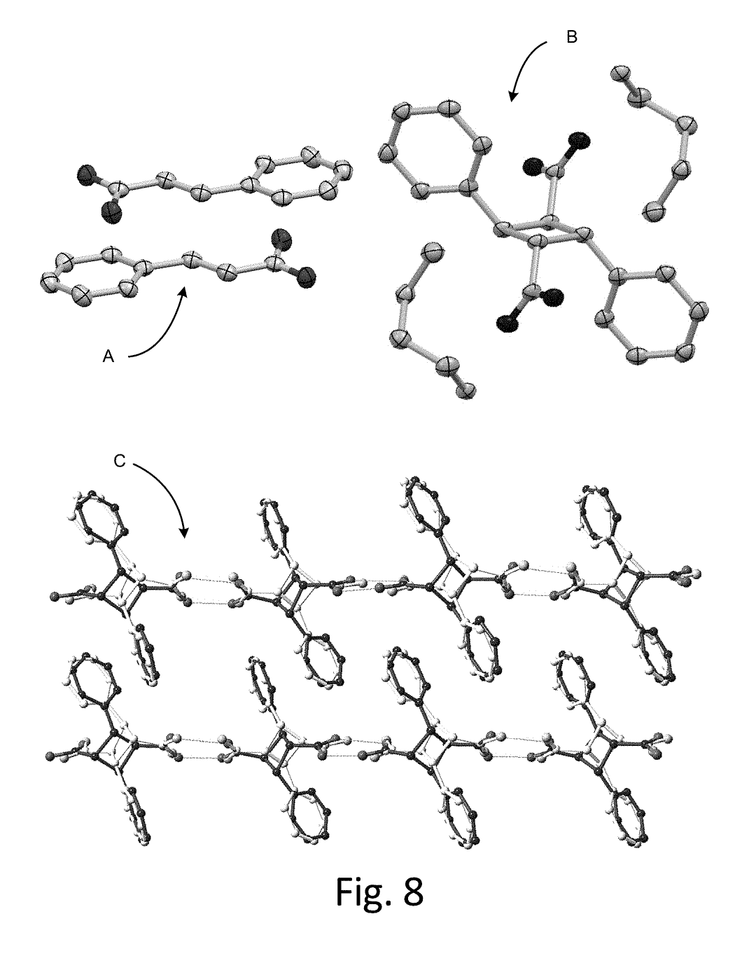

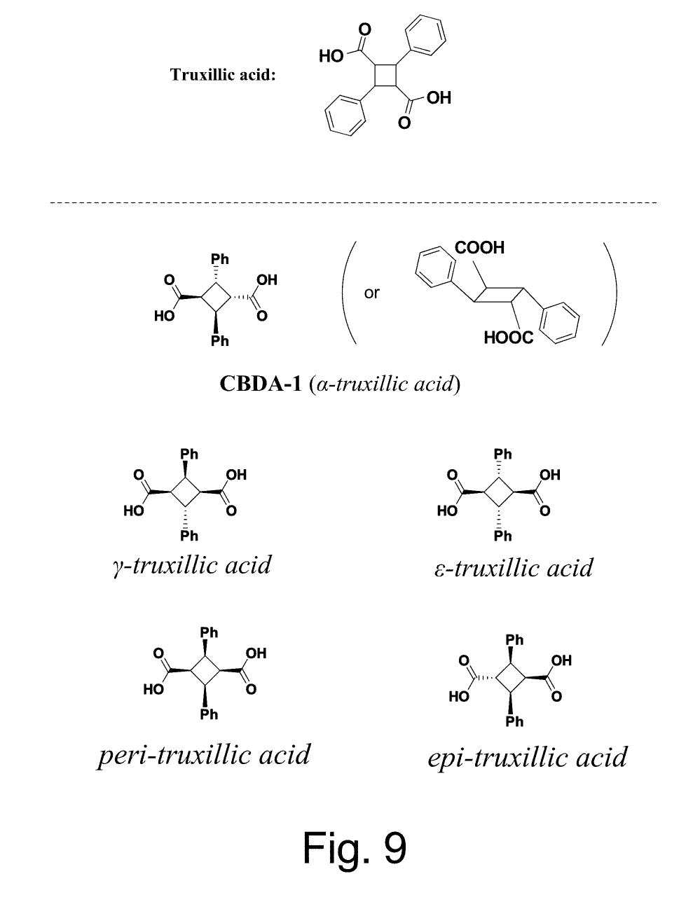

FIGS. 8-9 show more detailed structures of CBDA-1 molecules. FIG. 8 shows ORTEP representation of CBDA-1 at 50% electron density, while FIG. 9 shows chemical structures of CBDA-1 (truxillic acid) stereoisomers. FIG. 8 shows ORTEP representation of CBDA molecules at 50% electron density, including the crystal structure of trans-cinnaminc acid, CBDA-1 dibutylamine salt, and crystal form CBDA-1. FIG. 9 shows CBDA-1 and its five possible stereoisomers, including truxillic acid.

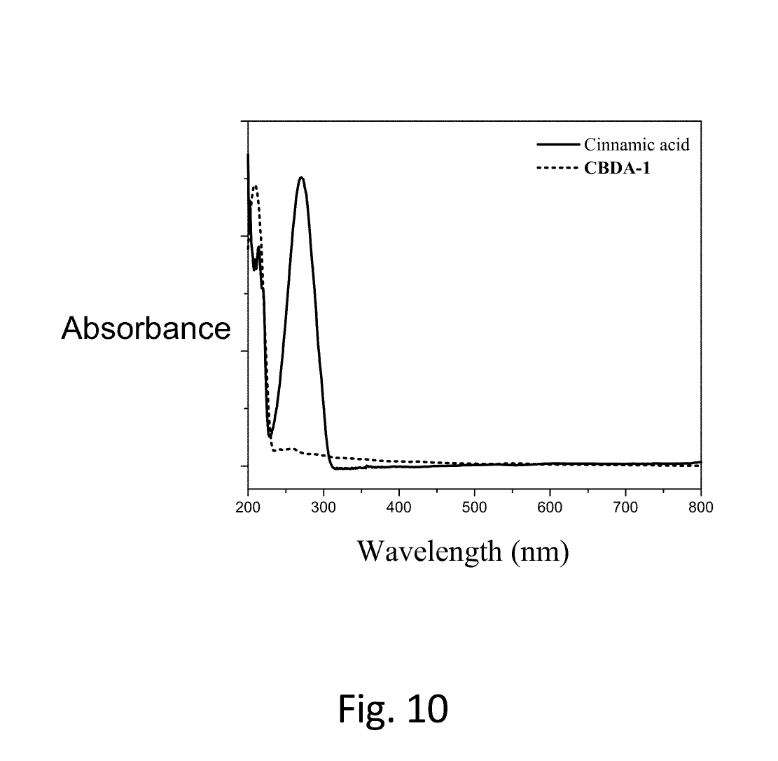

FIGS. 10-24 show spectra from testing of multiple embodiments of CBDA-1 polymers and poly-esters exhibiting properties of those molecules. FIG. 10 shows UV-Vis spectra of cinnamic acid and CBDA-1 molecules.

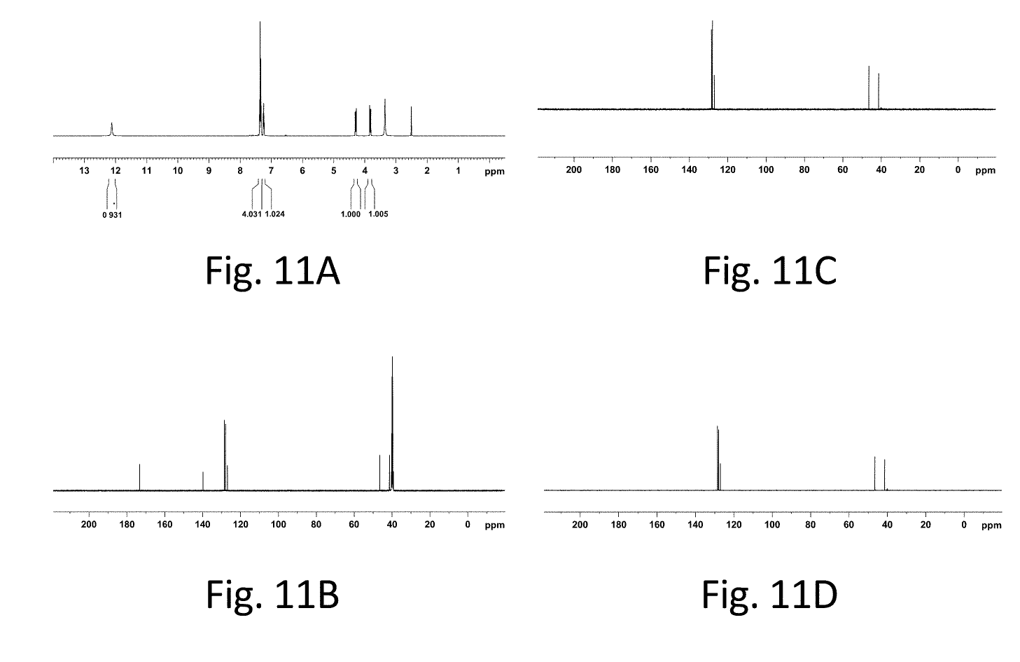

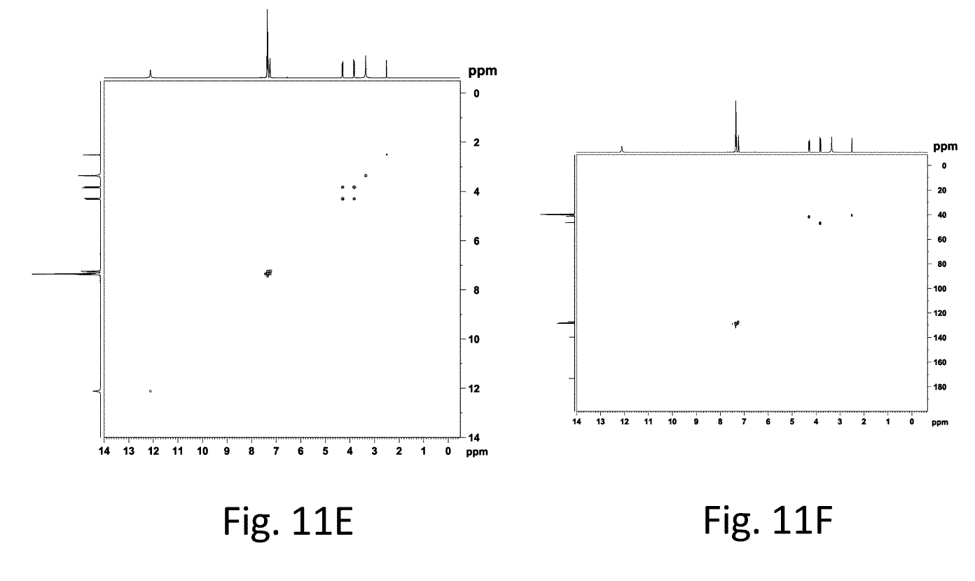

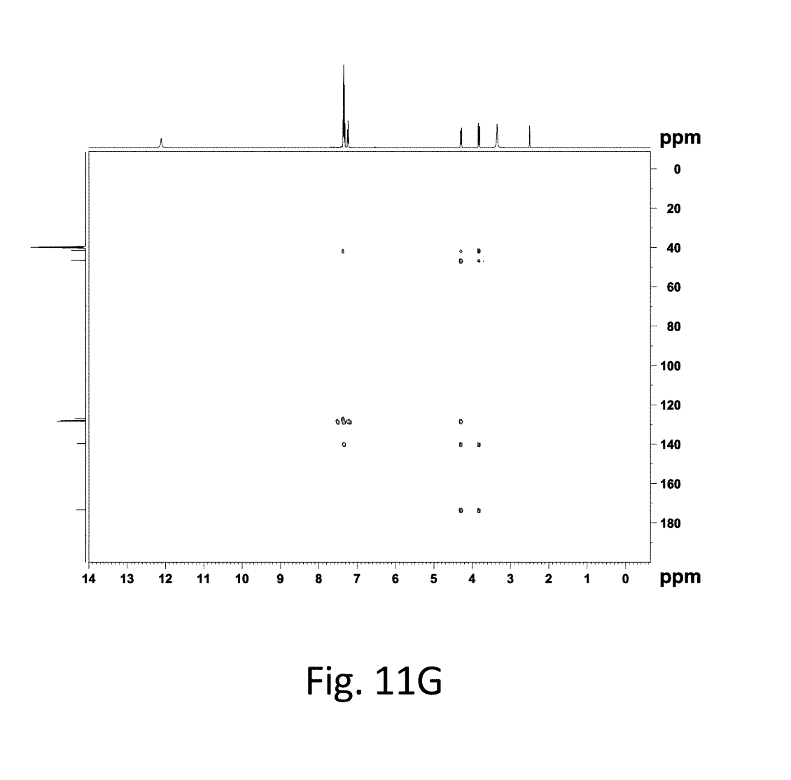

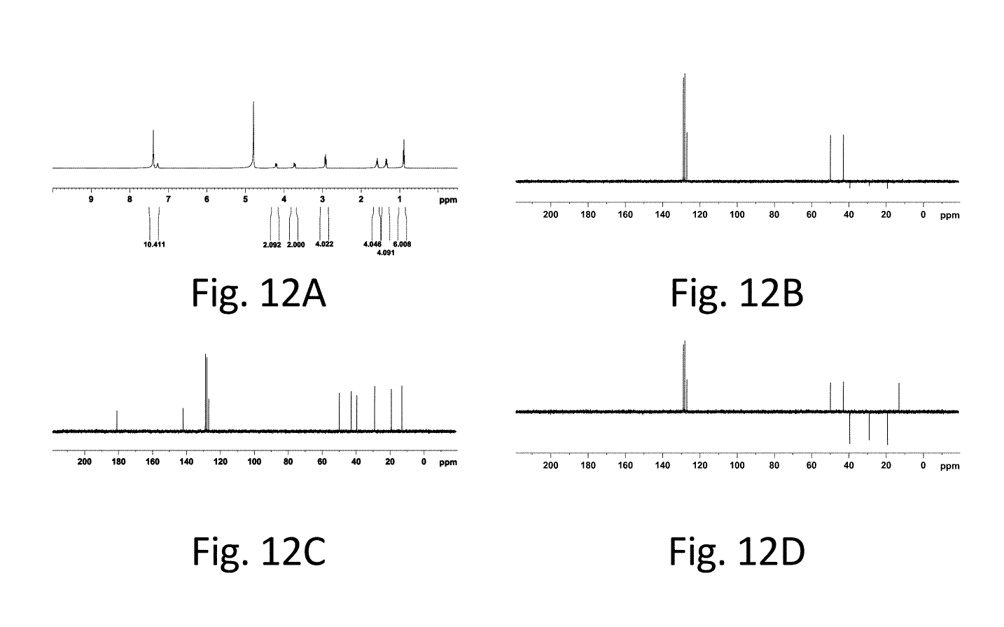

FIGS. 11-16 show NMR results from multiple embodiments of CBDA polymers and poly-esters created in lab. FIGS. 11A-11F show NMR spectra of CBDA-1. In order, they show ¹H-NMR, ¹³C-NMR, DEPT 135, 90 ¹³C NMR, COSY NMR, HSQC NMR, and HMBC NMR of α-truxillic acid in DMSO-d₆ at room temperature. FIGS. 12A-12D show ¹H, ¹³C, DEPT 90 and ¹³C DEPT 135 NMR spectra of another embodiment, α-truxillate-dibutylaminium in D₂O at room temperature.

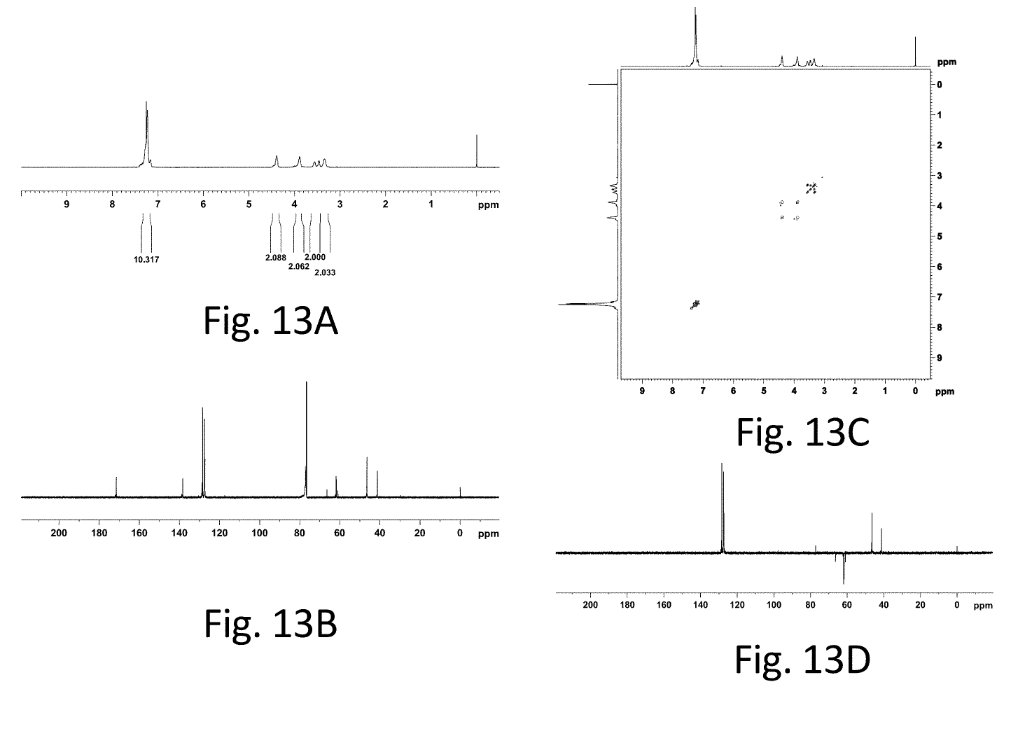

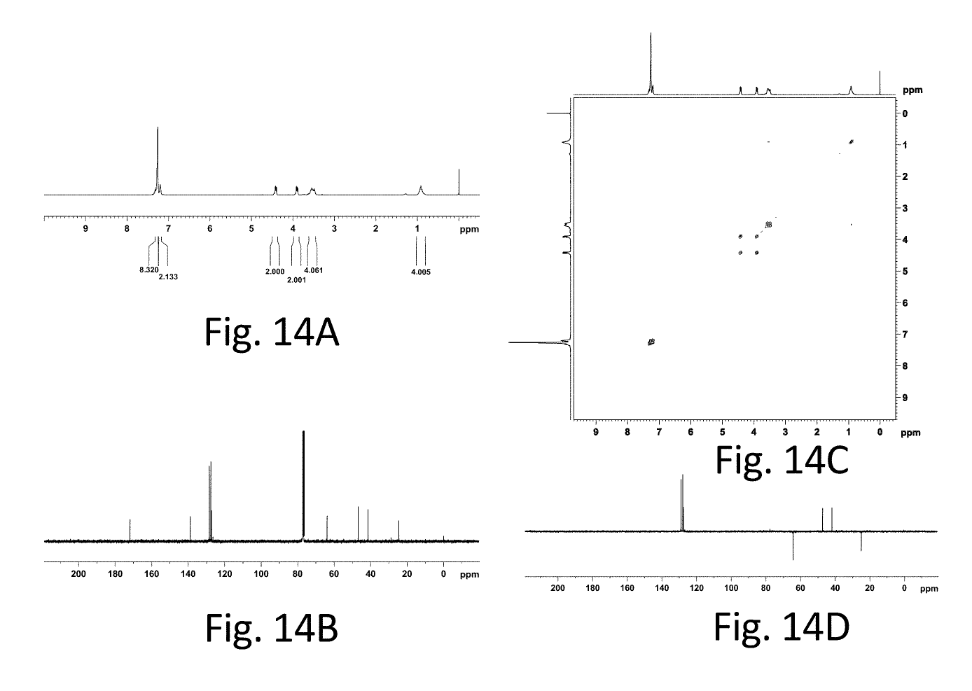

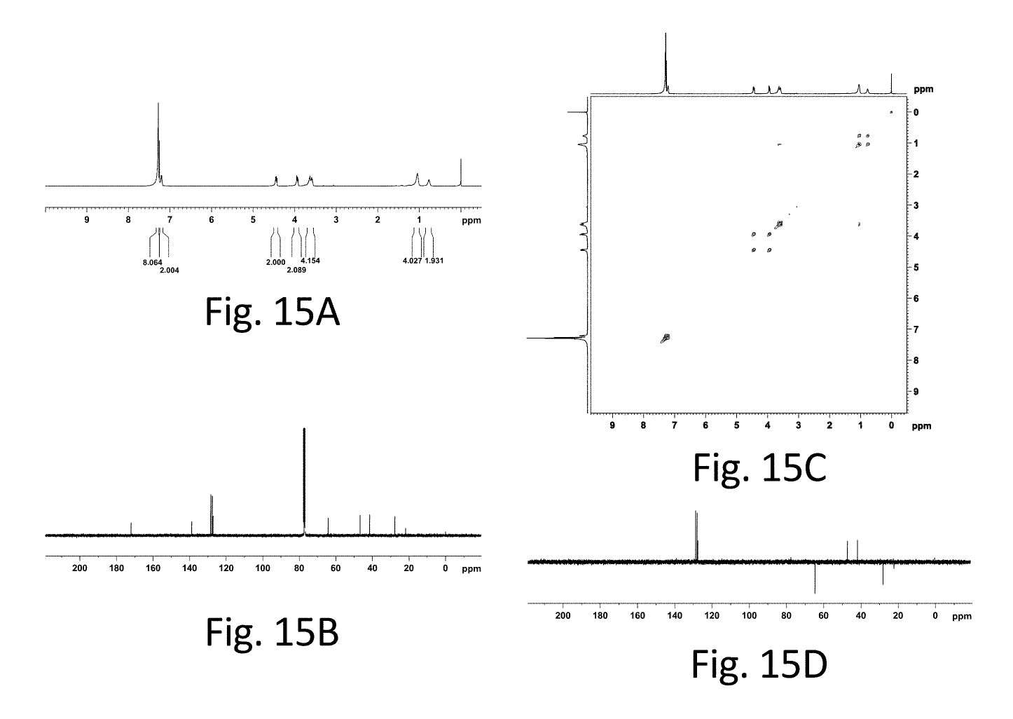

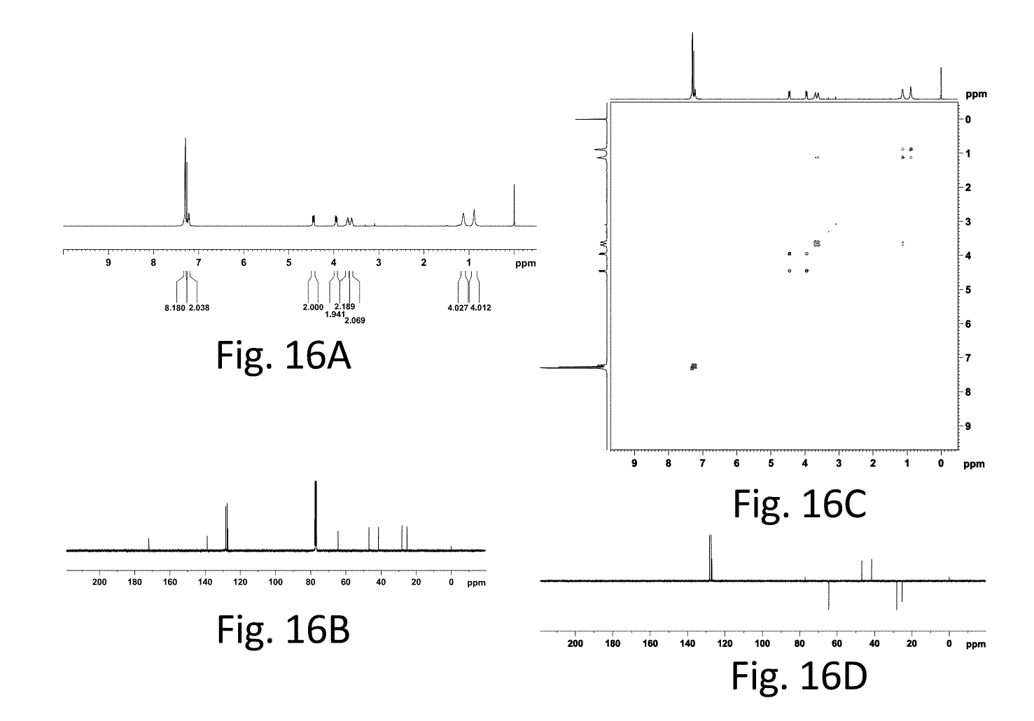

FIGS. 13-16 show NMR spectra of various embodiments of a CBDA polymer. FIGS. 13A-13D show ¹H, ¹³C, ¹H, COSY and ¹³C DEPT 135 NMR spectra of another embodiment, PEAT (poly(ethylene-α-truxillate)). FIGS. 14A-14D show ¹H, ¹³C, ¹H, COSY and ¹³C DEPT 135 NMR spectra of another embodiment, PBAT (poly(1,4-butylene-α-truxillate)). FIGS. 15A-15D show ¹H, ¹³C, ¹H, COSY and ¹³C DEPT 135 NMR spectra of another embodiment, PPAT (poly(1,5-pentylene-α-truxillate)). FIGS. 16A-16D show ¹H, ¹³C, ¹H, COSY and ¹³C DEPT 135 NMR spectra of another embodiment, PHAT (poly(1,6-hexylene-α-truxillate)).

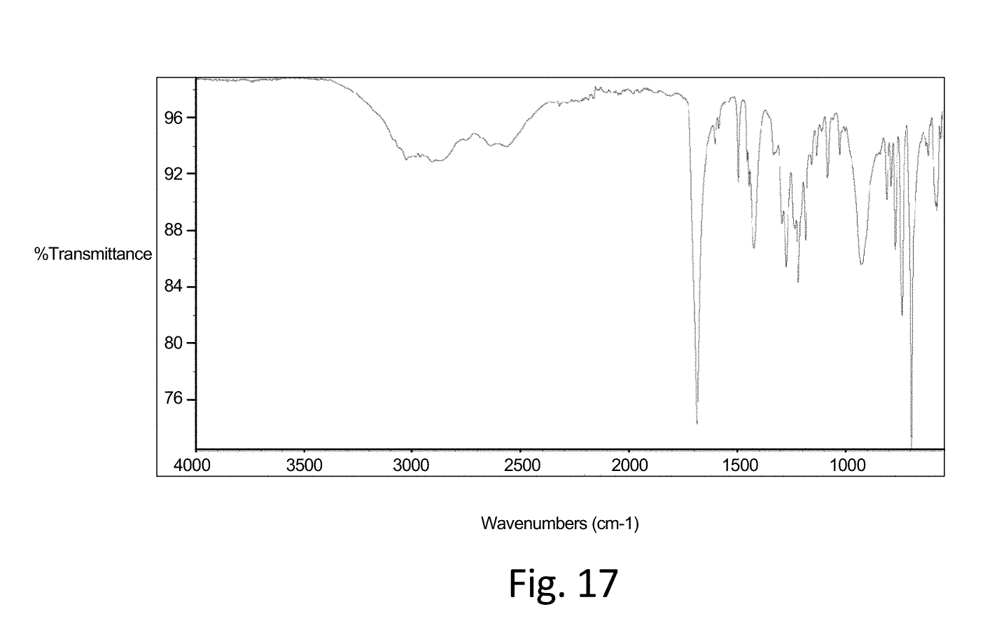

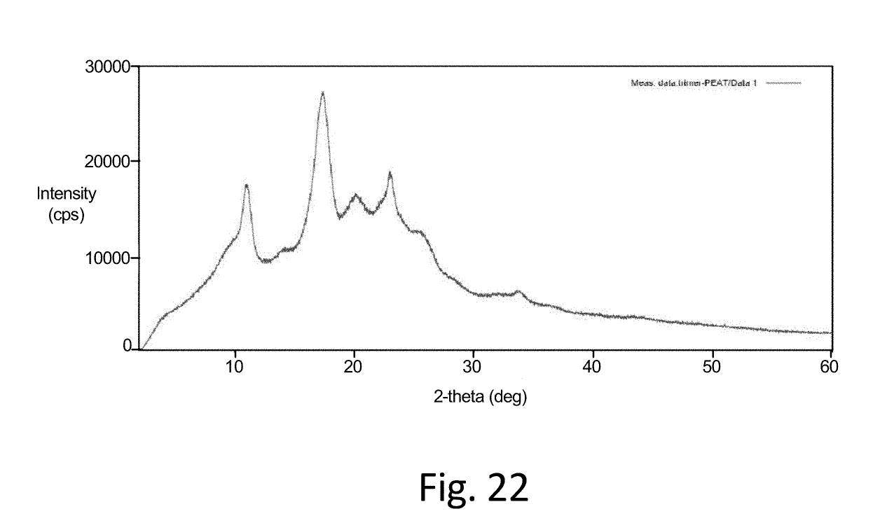

FIGS. 17-21 show FT-IR spectra for multiple embodiments of CBDA polymers. FIGS. 17 shows the FT-IR spectra of CBDA-1 for reference. FIG. 17-21 show the FT-IR spectra for varying embodiments of CBDA-1 polymers, in order, PEAT, PBAT, PPAT, and PHAT. FIGS. 22-25 show powder X-ray diffraction of multiple embodiments of CBDA polymers. In order, they show powder X-ray diffraction plots for PEAT, PBAT, PPAT and PHAT.

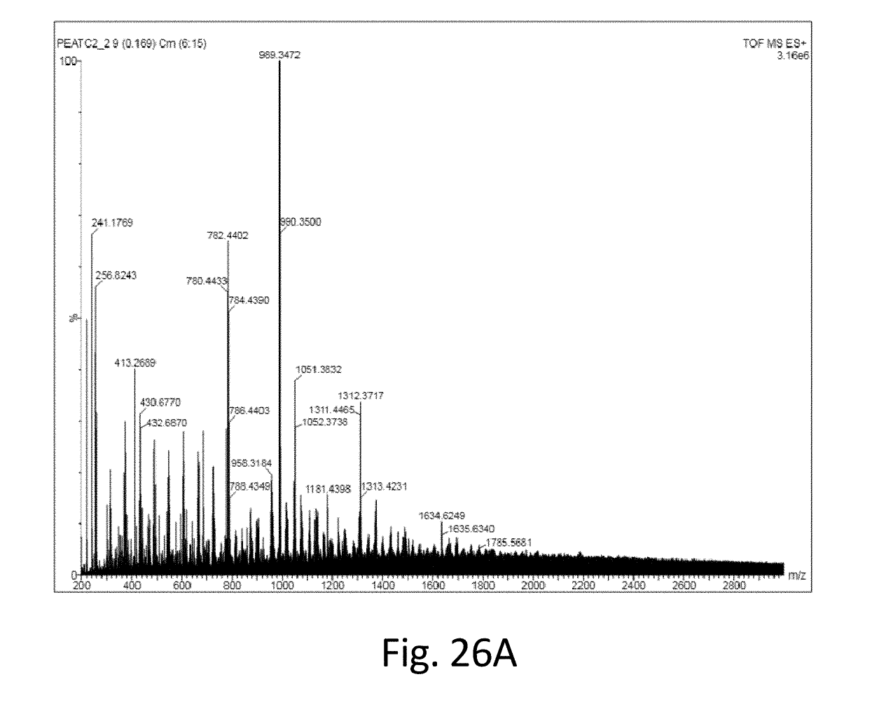

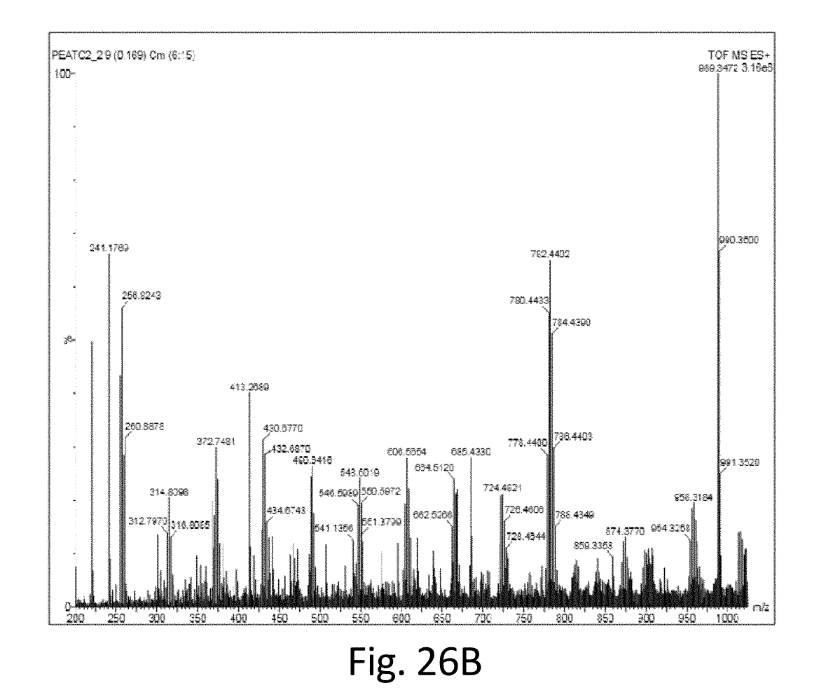

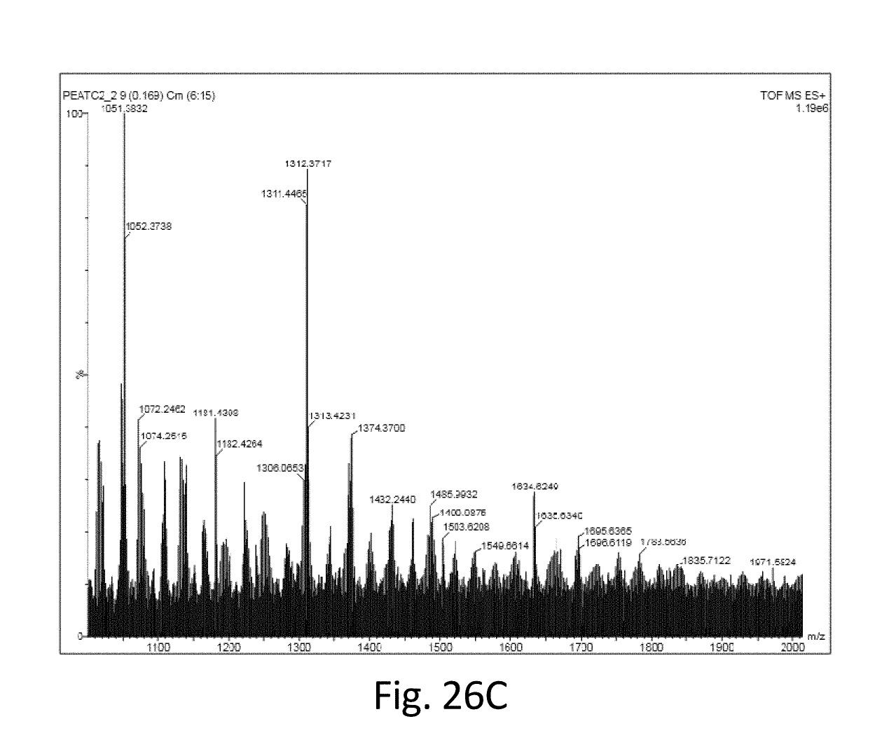

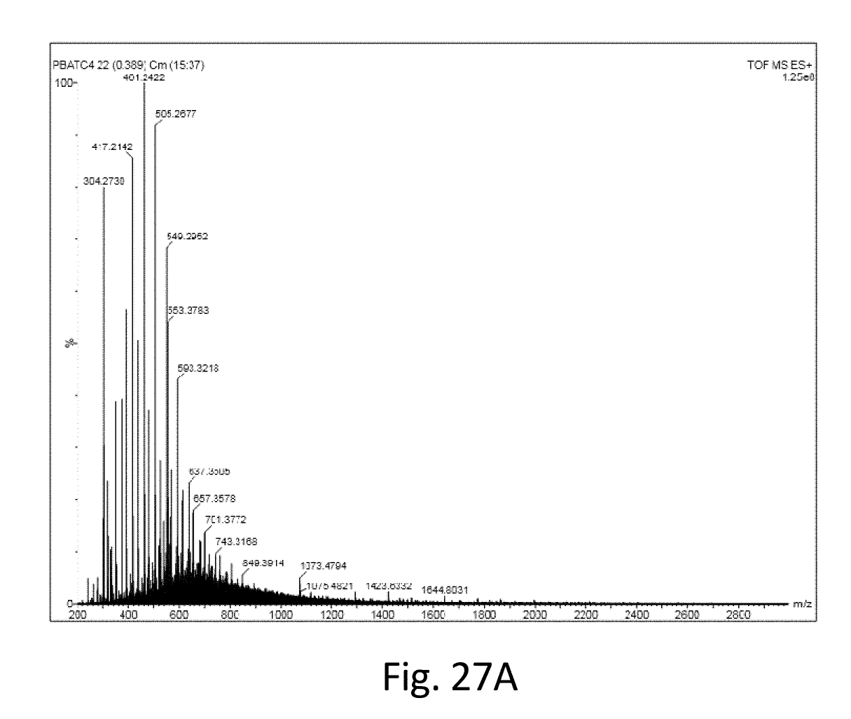

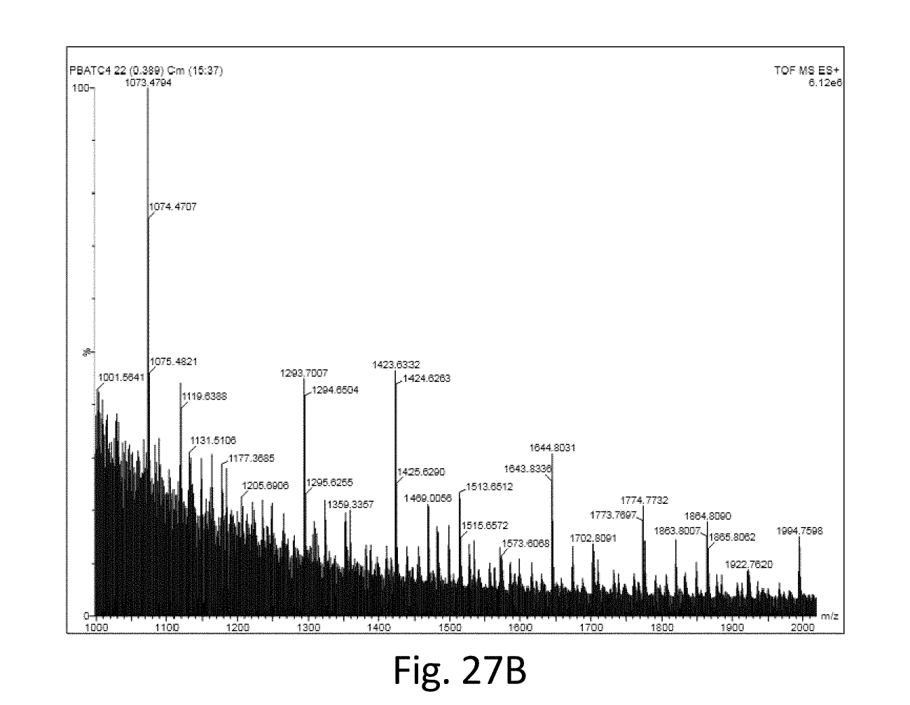

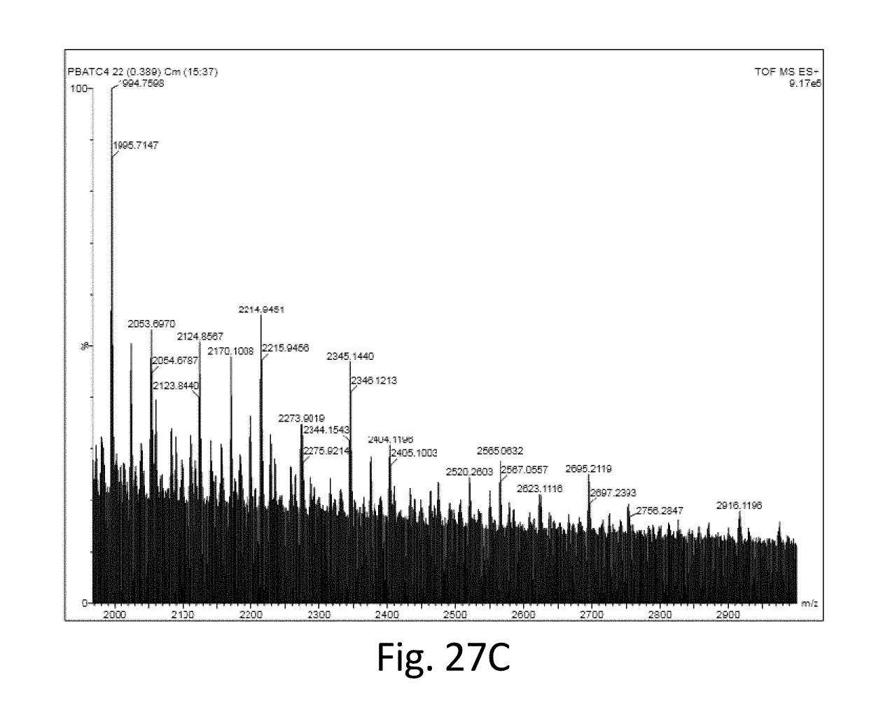

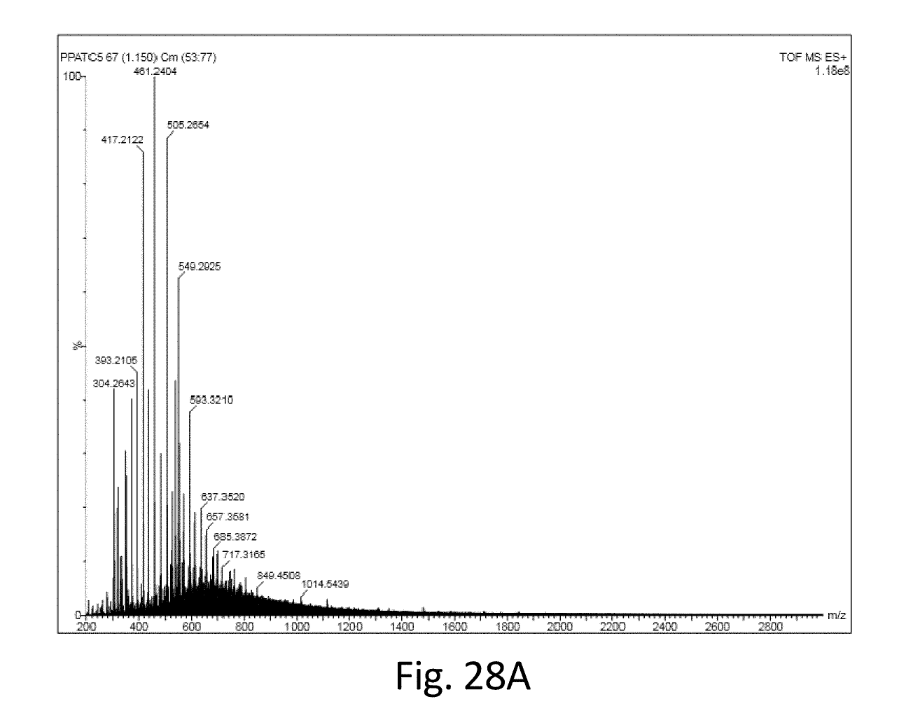

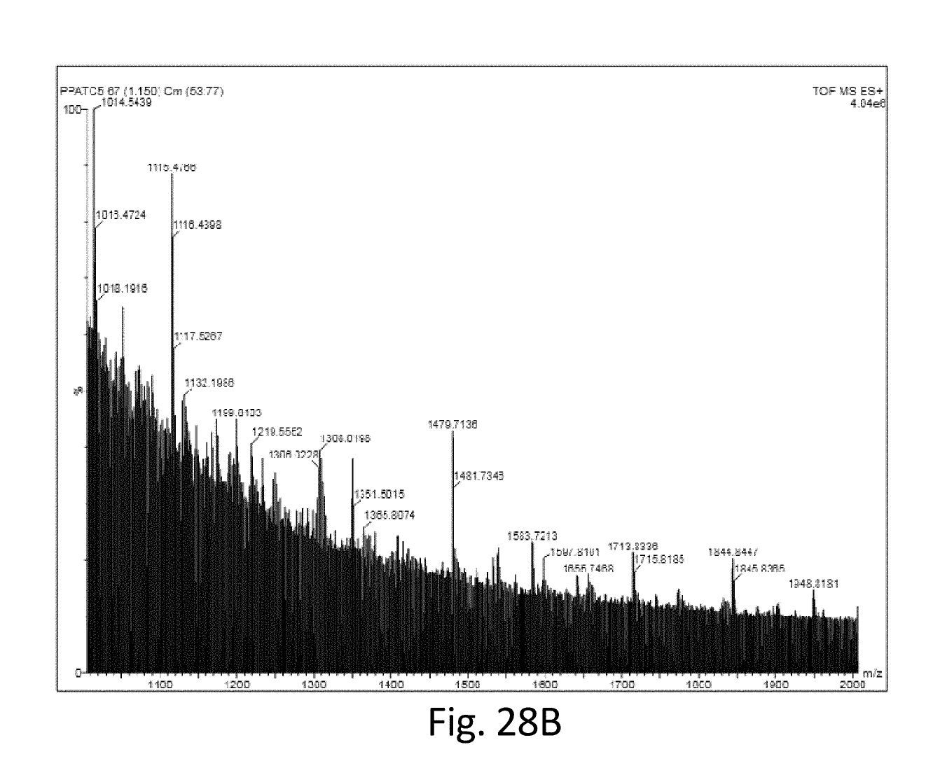

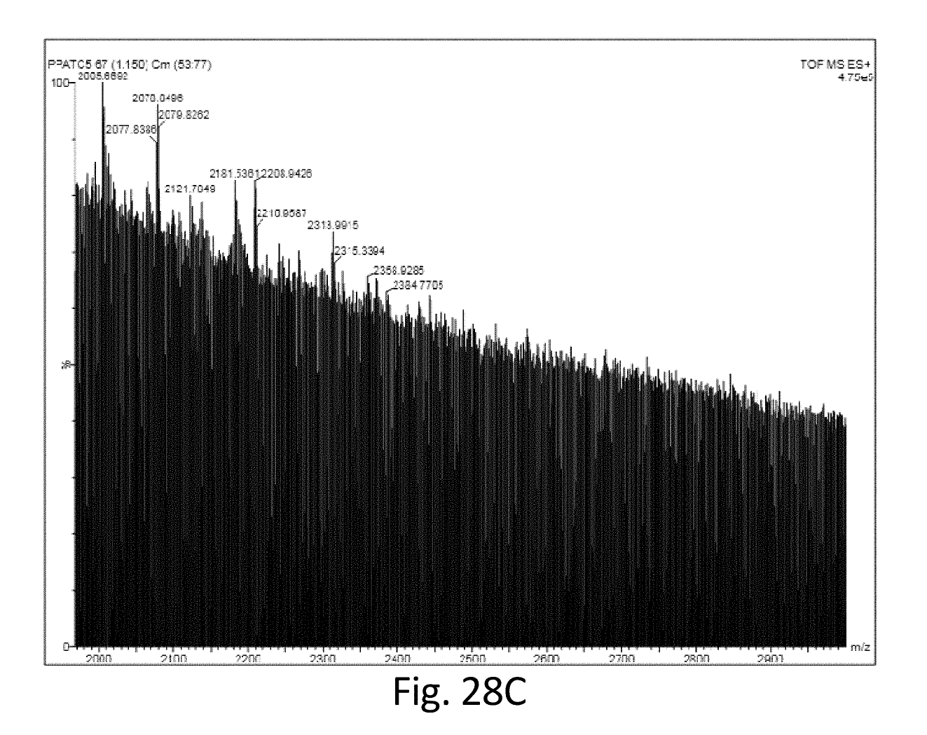

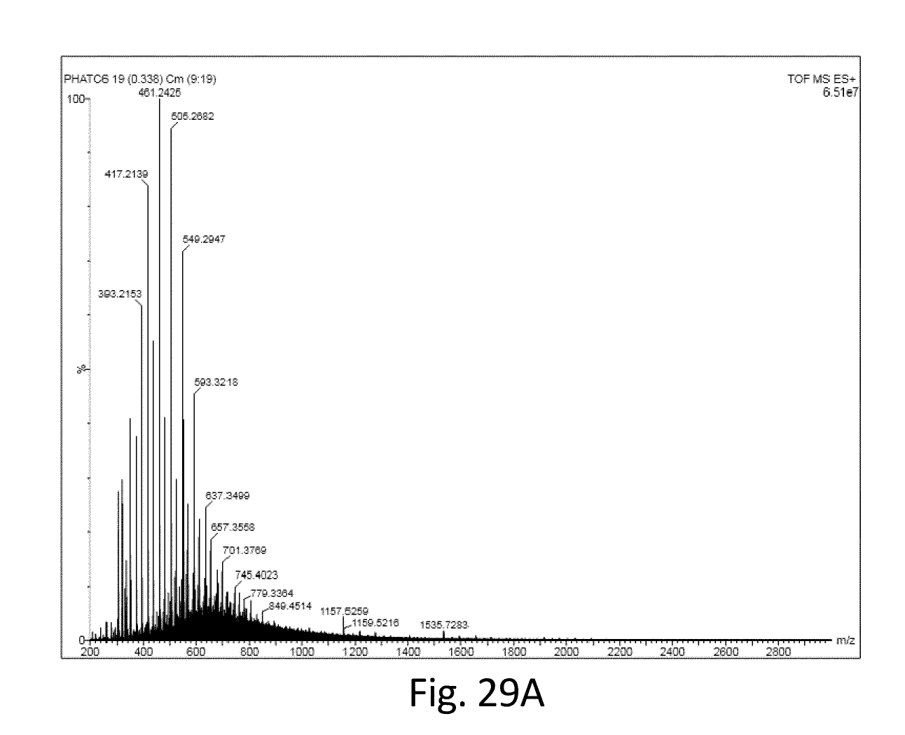

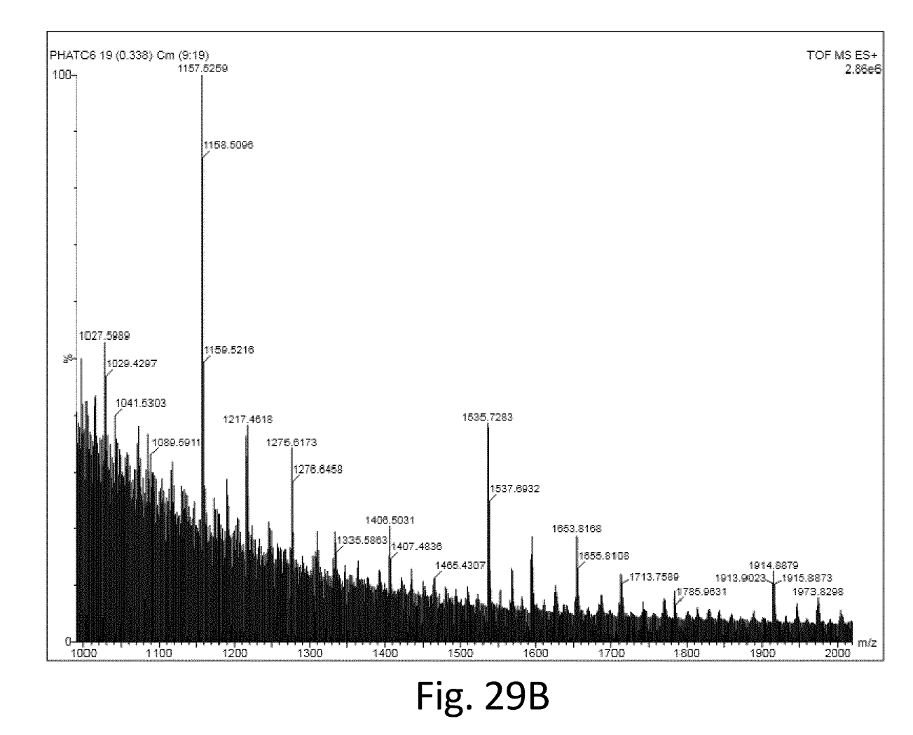

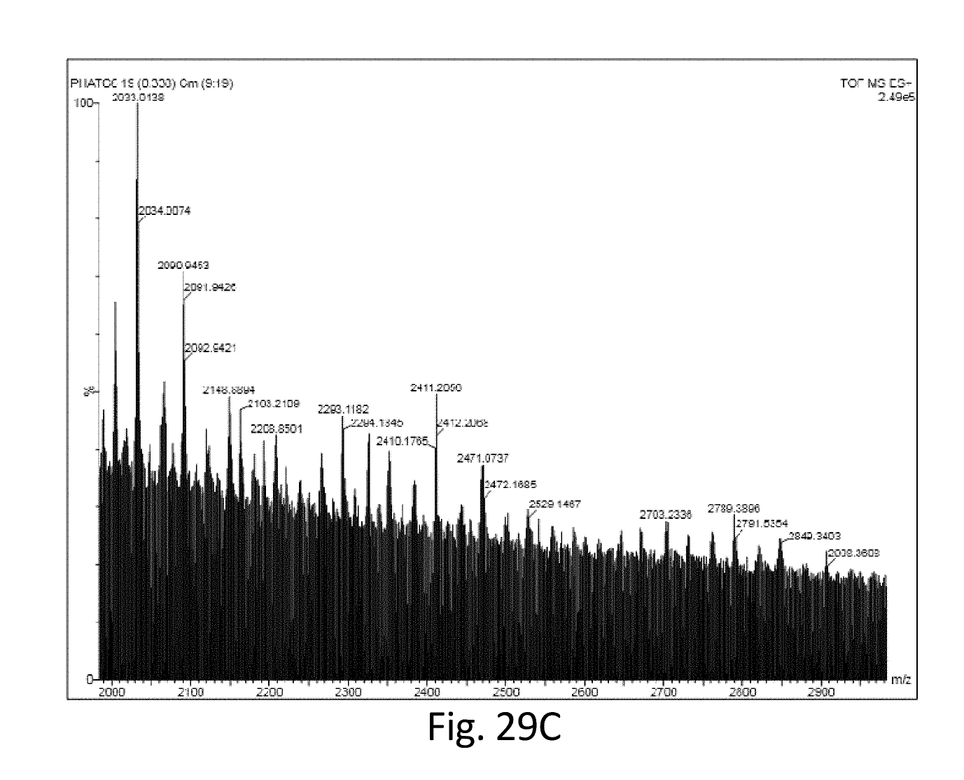

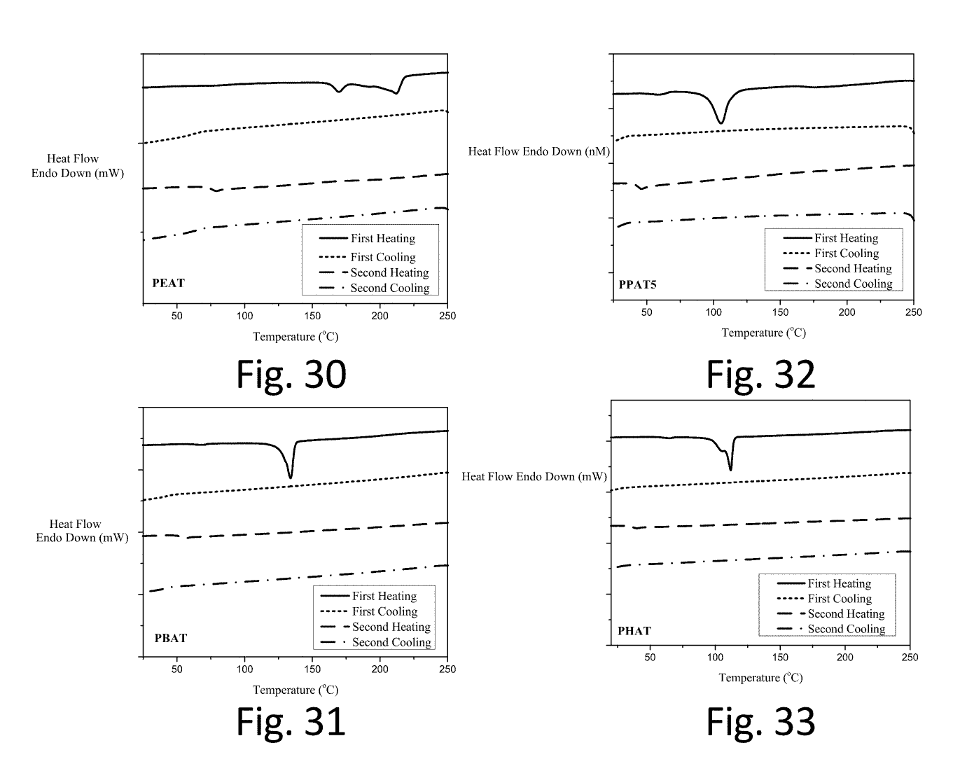

FIG. 26-29 show MS spectra of multiple embodiments of CBDA polymers. FIGS. 26A-26C show MS spectra of PEAT; FIGS. 27A-27C show MS spectra of PBAT; FIGS. 28A-28C show MS spectra of PPAT; and FIGS. 29A-29C show MS spectra of PHAT. FIGS. 30-33 show DSC results of multiple embodiments of CBDA polymers going through first and second heating and cooling. In order, FIGS. 30-33 show curves for PEAT, PBAT, PPAT, and PHAT.

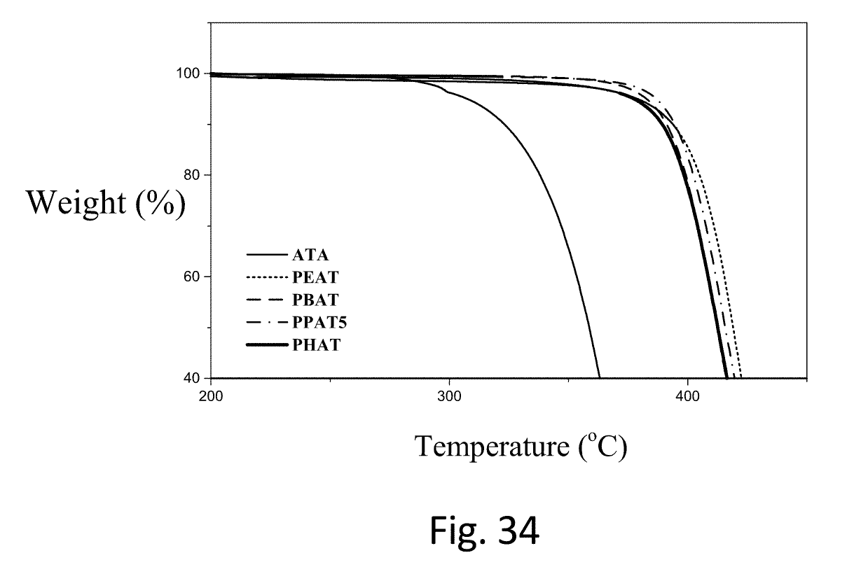

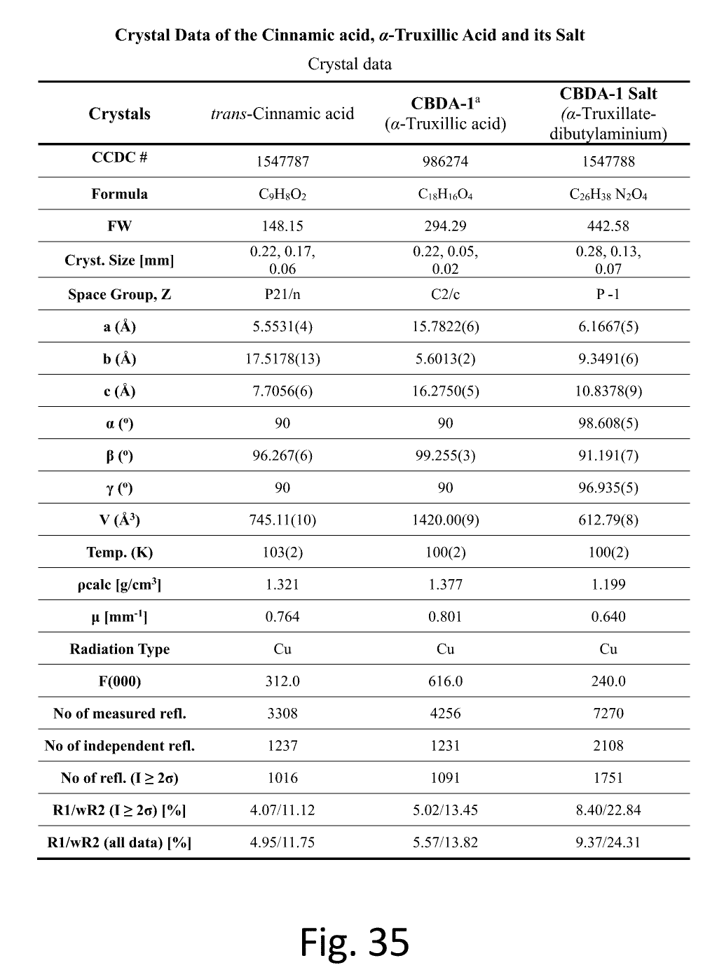

FIG. 34 shows TGA of multiple embodiments of CBDA polymers under nitrogen (50 mL/min) with a heat rate of 20° C. per minute. FIG. 35 is a table showing crystal data of multiple embodiments of CBDA polymers, and summarizes many results shown in earlier spectra.

### Synthesis of CBDA-1

Prior to synthesis of polymers with CBDA, a usable CBDA monomer must be synthesized. (1α,2α,3β,4β)-2,4-diphenyl-1,3-cyclobutanedicarboxylic acid (CBDA-1) can be readily synthesized from commercially available trans-cinnamic acid via photodimerization in the solid-state. This process can be fulfilled in 8 hours in near quantitative yield without side products, allowing CBDA-1 to be used in subsequent steps without further purification.

The efficiency of the solvent-free photoreaction is due to complementary π-π interactions between adjacent trans-cinnamic acid molecules, which are enabled or potentiated by head-to-tail (α-form) packing in the solid state. Phenyl groups, on one end of the molecule, act as weak electron donating groups while carboxylic acid groups, on the opposite end, function as weak electron accepting groups. The end result is that, flat, conjugated trans-cinnamic acid molecules are relatively polar and prefer a head-to-tail packing formation because it is lower in energy.

High quality single crystals of trans-cinnamic acid were obtained in a mixed solvent of ethyl acetate and acetonitrile (1:1). X-ray diffraction analysis confirmed its head-to-tail packing. The head-to-tail packing can be obtained in a variety of solvents, including acetonitrile, acetone, toluene, methanol, tetrahydrofuran (THF), and chloroform showing that the α-form is the dominant packing conformation for trans-cinnamic acid. Moreover, powder X-ray diffraction (PXRD) confirms that the packing of commercial trans-cinnamic acid is the head-to-tail form because its powder pattern is nearly identical to that of the head-to-tail single crystal simulation. Consequently, commercial trans-cinnamic acid powder can be used to produce the building block, CBDA-1, directly without recrystallization. Only one of the five stereoisomers of the [2+2] head-to-tail dimers was produced because solid state photoreaction normally proceeds with minimum movement of atoms.

### Thermostability, Chemical and Photochemical Stability Analysis of CBDA-1

The synthesized CBDA-1 monomers were thermally, chemically, and photochemically stable when analyzed. As shown in FIGS. 3A-3B, Thermogravimetric analysis (TGA) and differential scanning calorimetry (DSC) studies show CBDA-1 was stable at 250° C. and only loses 5% of its weight when the temperature is increased to 307° C. Weight lost in TGA below 330° C. may be due to evaporation because CBDA-1 melts around 276° C. and it lost all weight at 378° C. showing its decomposition at high temperature. A DSC curve suggested CBDA-1 started decomposing at 330° C. in the first heating process. After decomposition, DSC showed no change in the first cooling and second heating processes. The TGA and DSC results showed that CBDA-1 is thermally stable enough to be used as a building block in materials.

After exposure of CBDA-1 under sunlight for a month, a CBDA-1 sample showed no change in color or in its proton NMR spectrum as shown in FIGS. 11A-G. Comparison of the UV-Vis spectra of CBDA-1 and its starting material trans-cinnamic acid showed a clear blue shift with the maximum absorption moving from 270 nm to 208 nm after photoreaction as shown in FIG. 10. The formation of two sp³ carbons between the phenyl ring and carboxylic acid group led to the de-conjugation, which is responsible for the shift and sunlight stability of the CBDA-1.

In addition to thermal and sunlight stability, the cyclobutane ring of CBDA-1 also shows good chemical stability. No change was observed after boiling CBDA-1 in 6 M HCl at 100° C. for 24 hours and its ¹H NMR spectrum confirmed that there was no isomerization or any other changes (see FIGS. 11A-G). As expected, CBDA-1 reacted with KOH via acid-base neutralization reaction. After boiling CBDA-1 in 15 M KOH aqueous solution for 24 hours, the mixture was acidized with HCl to pH=3 and CBDA-1 was precipitated out and filtered. Its ¹H NMR showed no change.

### Single Crystal Structure X-ray Diffraction Analysis of CBDA-1

Cyclobutane rings in the structure adopted two different orientations which were randomly dispersed throughout the crystal matrix, resulting in a disordered structure. This may have occurred because the relatively small cyclobutane ring did not fully fill the empty space generated in the crystal matrix by linear hydrogen bond chain and the relatively rigid structure of CBDA-1 as shown in FIGS. 4A-4C and FIG. 8B-8C. straightforward single crystal structure was obtained by preparing a CBDA-1 salt, α-truxillate-dibutylaminium, in the solvent solution ethanol/water/dimethylformamide (1:1:1). The short-chain flexible cation, butyaminium does not destroy the structure of CBDA-1, but can be used to fill spaces in the crystal lattice reducing the chance of disorder.

The single crystal of CBDA-1 salt shows that the four carbon atoms on the cyclobutane ring are coplanar with carbon-carbon bond distances of around 1.57 Å. The two carboxylic groups on opposite sides of the cyclobutane ring, have a 180° angle between them and are offset by 1.40 Å, which is a unique characteristic compared to other well-known diacids. The distance between the two carboxylic groups is 4.76 Å. This distance is similar to the distance between two carboxylic groups on furan-based building block 2,5-furandicarboxylic acid as shown in FIG. 1. The spatial orientation and distance of the two carboxyl acid groups makes it suitable for polymer construction. The cyclobutane ring has two exchangeable conformations, planar and puckered with about 23 degrees difference between them. The limited conformational freedom of the cyclobutane ring is expected to give CBDA-1 a unique semi-flexible and semi-rigid character.

### Synthesis of CBDA Polymers, Poly-α-Truxillate, with CBDA-1

A CBDA-1 polymer, poly-α-truxillate, can be synthesized using a series of linear diols and condensation reactions to polymerize CBDA-1. The synthesis is pictured in FIG. 5. Five poly-α-truxillate can be produced, poly(ethylene-α-truxillate) (PEAT), poly(propylene-α-truxillate) (PPAT3), poly(l,4-butylene-α-truxillate) (PBAT), poly(1,5-pentylene-α-truxillate) (PPAT), poly(1,6-hexylene-α-truxillate) (PHAT). Out of the five polymers synthesized, only PPAT3 is a liquid at room temperature, so it is not included in the following discussion.

Powder X-ray diffraction patterns of these four poly-α-truxillates showed that they are semi-crystalline as shown in FIGS. 22-25. Two series of peaks were observed in the HRMS spectrum of PEAT as shown in FIG. 6, which was determined by preliminary MALDI-TOF analysis. They have a repeating unit with m/z=322.12 that corresponds with the unit mass of PEAT (C₂₀H₁₈O₄ m/z=322.12). One repeating peak of PEAT is ‘m/z=322.12×n+22.99 (Na)’ which may indicate that some cycled polyesters exist in the product. For example, ‘m/z=322.12×3+22.99=989.35’. Another repeating peak of PEAT is ‘m/z=322.12×n+62.04 (end-group)+22.99 (Na)’. This result suggests there are linear polyesters with end-groups HO—(CH₂)₂— and —OH. For example, ‘m/z=322.12×3+62.04+22.99=1051.39’.

The MS analysis of PEAT revealed both linear and cycled fragments can potentially be present in PEAT, as shown in FIGS. 26A-26C. This phenomenon of two different repeating peaks in HRMS spectra was also observed in PBAT, PPAT, and PHAT (FIGS. 27-29). Both NMR and HRMS spectra indicated that PEAT was the only compound with a significant number of cycled products (FIGS. 12-16). This is probably because it is difficult to form cycled products when the linker molecules contain long and flexible carbon chains. The maximum molecular weights observed in HRMS spectra indicate six-mers.

Thermogravimetric analysis (TGA) indicates that the synthesized poly-α-truxillate have excellent thermal stability, which is consistent with its CBDA-1 building block as shown in FIG. 34. DSC was used to analyze the glass transition temperature (T_{g}) of the four polyesters as shown in FIGS. 30-33. The figures show a decreasing trend of the poly-α-truxillate T_{g}s with increasing diol carbon chain length. T_{g} of PEAT is 81° C. whereas T_{g} of PPAT is 64° C. This trend of the T_{g}s may be attributed to the increased flexibility of carbon chains with higher carbon numbers. It is easier for the polyester to rotate or twist with long carbon chain. After the first heating and cooling process, the DSC curve showed an obvious decreasing trend of T_{g}s in the second heating process. This phenomenon may be due to the annealing effect of the first heating process. After heating polyesters to 250° C., the polyesters will be scattered and mixed equally that may lead the decreasing T_{g}s.

### Discussion of CBDA-1 Results

The produced CBDA polymers (poly-α-truxillate) are unique compounds with a variety of industry applications where polymers or polyesters are typically used, including materials synthesis. The required starting materials for CBDA polymers are readily available. Both the diols and cinnamic acid can be obtained from biomass. While diols have been widely used in making plastics, cinnamic acid has found its applications in flavors, perfumes, synthetic indigo, and certain pharmaceuticals. It has recently been adequately derived from a side product of biofuel manufacture and from other renewable substrates including glucose via engineered E. coli. Biomass derived cinnamic acid has also been reported to be used for styrene synthesis through a decarboxylation reaction to produce environmentally friendly polystyrene. Thus, CBDA-1 can potentially be made as a bio-based diacid to replace or partially replace the commodity petrochemicals such as terephthalic acid in the future.

In synthesis of CBDA polymers, a variety of non-reactive R groups and reactive X-groups can be used to link together CBDA monomers. The X groups of the linker molecule react with the carboxylic acids on the CBDA-1 molecules, and polymerize the CBDA-1 molecules in the produced polymers. A variety of linker molecules can be used to produce varying types of polymers and polyesters from CBDA-1. These variants are discussed in more depth below under “Modification of CBDA Polymers.”

Moreover, the synthesis of CBDA-1 monomers for use as polymer building blocks can be accomplished with ease, and produced stable compounds. The preparation of CBDA-1 has been discussed and rationalized. Using CBDA-1 in the construction of materials is not only beneficial because it can be produced from bio-based chemicals, but also because it has thermal, sunlight, and chemical stability.

Additionally, the four-membered carbon ring structure of CBDA offers a unique semi-flexible property for materials. These features of CBDA-1 allow it to be used directly in making new polymers or be added into known polymer receipts in certain ratio to modify their physical properties such as transparency and glass transition temperature.

The successful synthesis of a new family of polyesters presented show that CBDA-1 is a useful building block for polymers. TGA and DSC analyses revealed the thermal properties of the newly synthesized polyesters, which are comparable to the thermostability of PET. As a novel building block in materials, CBDA provides great opportunities in producing many materials (e.g., polyesters, polyamides, polycyclobutanes, copolymers, and coordination polymers) with new properties and applications (e.g., monomers, crosslinkers, and pharmaceutical precursors).

### II. Polymer Synthesis from CBDA-5

Alternatively, CBDA building blocks can be created from biomass components such as furfural. For instance, (1α, 2α, 3β, 4β)-2,4-di(furan-2-yl)cyclobutane-1,3-dicarboxylic acid (CBDA-5), a variant cyclobutane-containing dicarboxylic acid, can be created from furfural from a solvent-free photocycloaddition by using black light as an ECO-UV irradiation source. This semi-rigid diacid renewable building block and its ester exhibited thermal stability and chemical properties necessary for polymer formation. Thus, polymerization of CBDA-5 can also produce polyesters as an alternative to CBDA-1 produced from trans-cinnamic acid.

Similar to CBDA-1, the alternative CBDA-5 represents a unique semi-rigid building block in material synthesis due to the presence of the small aliphatic ring that can produce polyesters such as polyethylene cyclobutane-1,3-dicarboxylate (PEC-1) with sufficient thermal and chemical properties for industry use. CBDA-5 can be derived from waste material that can be turned into furfural, such as lignocellulose biomass. Thus, CBDA-5 is a green polymer building block alternative.

### Overview of CBDA-5 Figures

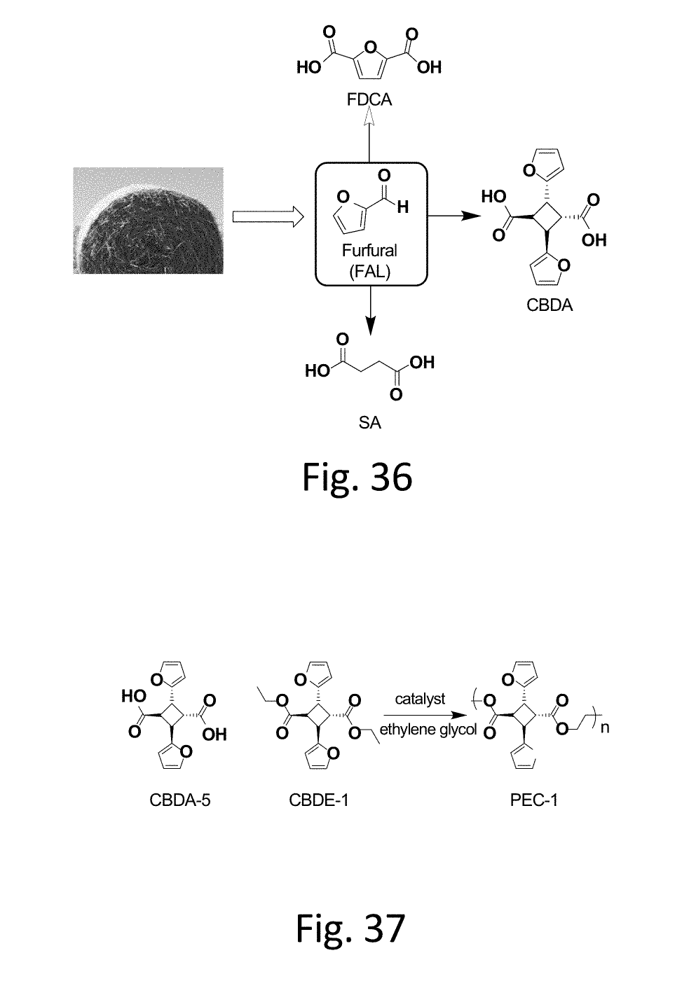

FIGS. 36-58 show laboratory results from production of CBDA-5 and CBDA-5 based polyesters. Various types of chemical analysis, including NMR, FT-IR, and MS spectra, TGA and DSC thermal analysis, and others were used to confirm that (1) CBDA-5 was produced, and the structure of CBDA-5 was as expected, (2) polyesters from CBDA-5 were produced and their structure. Overall, CBDA-5 is a useful, semi-rigid and thermally chemically stable monomer. For these reasons, CBDA-5 is a suitable building block for creating polyesters. Because CBDA-5 can be derived from lignocellulose biomass, CBDA-5 is also a green source of polymer building blocks.

FIG. 36 depicts renewal diacid polymer building blocks from furfural. Furfural (FAL) can be produced from biomass containing lignin. Furfural has previously been converted to succinic acid (SA) or furandicarboxylic acid (FDCA), both of which have previously been used for production of polyesters, such as polyethylene furanoate or polybutylene succinate. But furfural can also be converted to cyclobutane-1,3-dicarboxlyic acid (CBDA). Specifically, furfural can be converted into (1α,2α,3β,4β)-2,4-di(furan-2-yl)cyclobutane-1,3-dicarboxylic acid (CBDA-5) via photocycloaddition reaction. Alternatively, furfural can be converted to (1α, 2α, 3β, 4β)-2,4-di(furan-2-yl)cyclobutane-1,3 -diester (CBDE-1), the diester of CBDA-5.

FIG. 37 depicts a method of using CBDA-5 or CBDE-1 as a building block for polymer synthesis. The method of FIG. 37 uses a plurality of CBDA-5 or CBDE-1 molecules with a linker molecules and a catalyst to form a polymer, polyethylene cyclobutane-1,3-dicarboxylate (PEC-1). The synthesis of PEC-1 is similar to the process described with reference to FIG. 2 using CBDA-1 molecules. Like the first process, polymerization of CBDA-5 (or CBDE-1) requires linker molecules containing a non-reactive R group and a plurality of X groups that react with the carboxylic acid of the CBDA-5 (or CBDE-1) molecules.

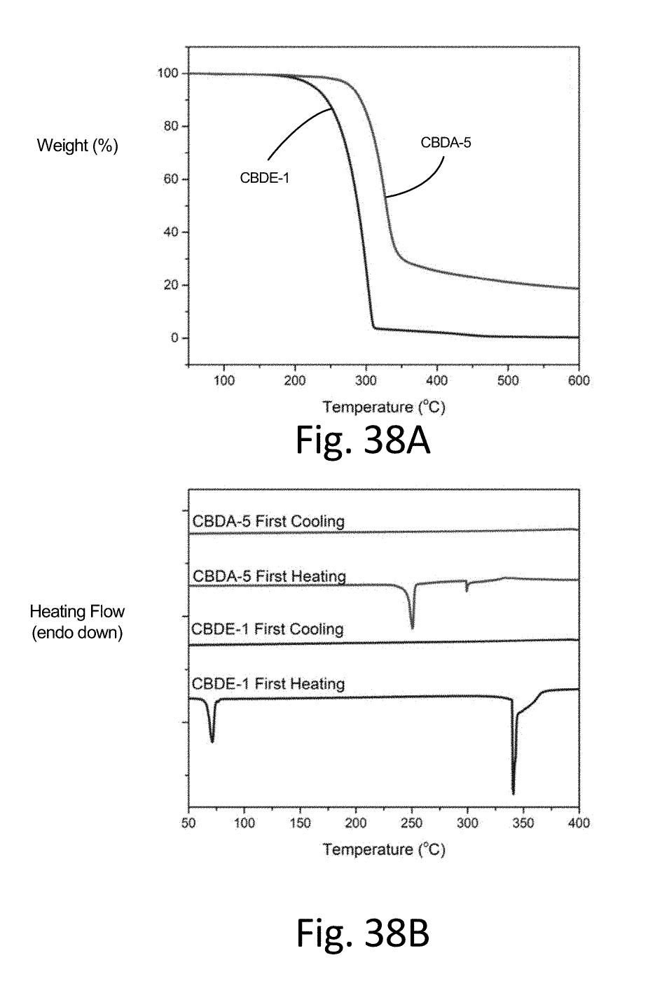

FIGS. 38A-38B show thermogravimetric analysis (TGA) and differential scanning calorimetry analysis (DSC) of CBDA-5 and CBDE-1 building blocks. FIG. 38A shows a thermogravimetric analysis (TGA) of CBDA-5 and CBDE-1 that was recorded from 50° C. to 600° C. with a heating rate of 20° C. per minute under a nitrogen atmosphere. CBDE-1 started decomposition at a lower temperature (approximately 183° Celsius) compared with CBDA-5 (approximately 202° Celsius). Both CBDA-5 and CBDE-1 had single stage decomposition and the maximal decomposition rates were at approximately 327° C. and 303° C., respectively.

FIG. 38B shows differential scanning calorimetry (DSC) analysis of CBDA-5 and CBDE-1 molecules. DSC was recorded from 0° C. to 400° C. with a heating rate of 10° C. per minute under a nitrogen atmosphere. The melting points of CBDA-5 and CBDE-1, respectively, were 73° C. and 235° C. CBDE-1 has a much lower melting point than CBDA-5, which can be attributed to the intermolecular hydrogen bonds between carboxylic acid groups of CBDA-5. CBDE-1 lacks these hydrogen bonds. The TGA and DSC curves were consistent, and analysis reveals that both CBDA-5 and CBDE-1 are thermally stable enough to be used as polymer building blocks.

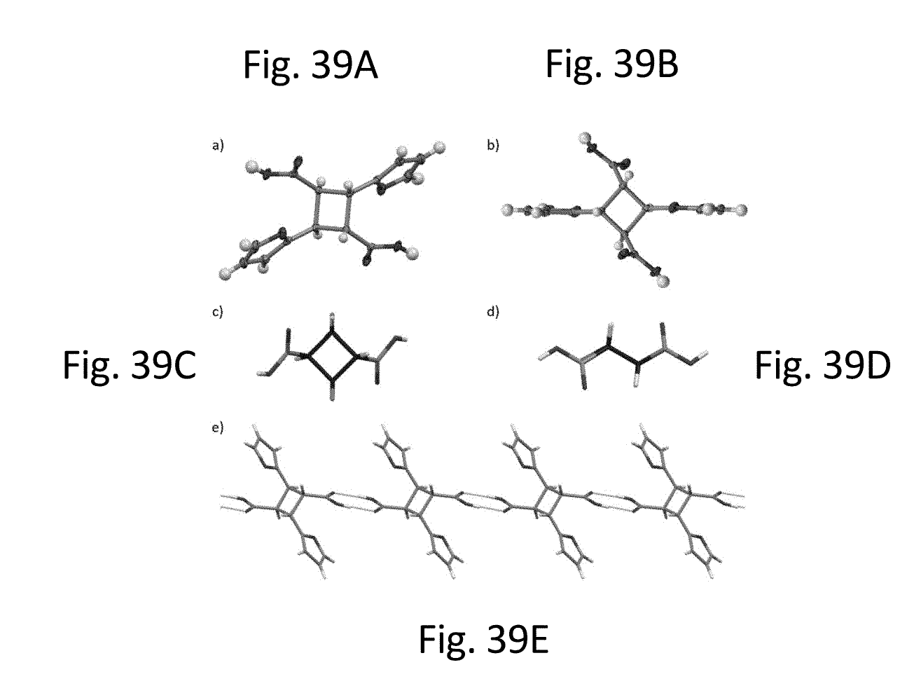

FIGS. 39A-39E picture the single crystal X-ray diffraction structure of CBDA-5 building blocks. FIGS. 39A and 39B show, respectively, the front view and side view of single crystal X-ray diffraction of CBDA-5. Here, the two furan groups are in a linear orientation.

FIG. 39C shows the top view of CBDA-5, which exhibits the square shape of the cyclobutane ring. X-ray single crystal analysis shows this cyclobutane ring is in a near planar configuration with a torsion angle of 1.5°. CBDA-5 exhibits exchangeable conformation between coplanar and puckered cyclobutane rings, meaning CBDA-5 maintains semi-rigid properties. Within the cyclobutane ring, the four interior angles are 89.2°, 90.8°, 89.2°, and 90.8°. The interatomic distance between the two carboxylic acid groups (about 4.70 Å) in CBDA-5 is shorter than the distance in furandicarboxylic acid (FDCA) (about 4.83 Å), also derived from furfural. Additionally, the two carboxylic acid groups in CBDA-5 are pointing in opposite direction than those in FDCA.

FIG. 39D shows the side view of CBDA-5, which is remarkably similar to the structure of succinic acid (SA) shown as a chemical structure in FIG. 36. However, the distance between the two carboxylic acid groups (about 4.70 Å) in CBDA-5 is longer than that of those in SA (about 3.85 Å). Because of the distances between the carboxylic acid groups and the angles at which they lay, this orientation of the carboxylic acids in CBDA-5 is suitable for building the backbone of a linear polymer.

FIG. 39E shows a hydrogen-bonded supramolecular complex of CBDA-5 molecules. This supramolecular complex shows the potential of preparing CBDA-5 into preparing supramolecular materials such as hydrogen bonded organic frameworks.

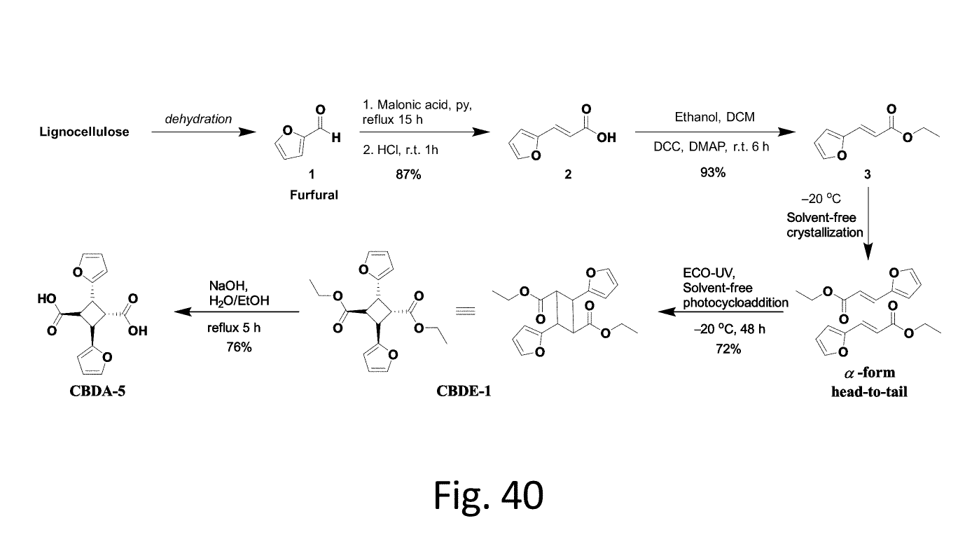

FIG. 40 shows synthesis of CBDA-5 and CBDE-1 from furfural. The preparation of furfural-based building blocks CBDA-5 and CBDE-1 can be started from malonic acid to create 2-furanacrylic acid (Compound 2) which can be dimerized through photocycloaddition reactions in the solid-state to produce (1R,2S,3R,4S)-rel-3,4-di(furan-2-yl)cyclobutane-1,2-dicarboxylic acid (CBDA-2). Low temperature solvent-free crystallization techniques can be used to prepare crystals of ethyl furanacrylate (Compound 3). Photocycloaddition can be used to produce CBDE-1. This methodology is discussed in more detail below under “Synthesis of CBDA-5.”

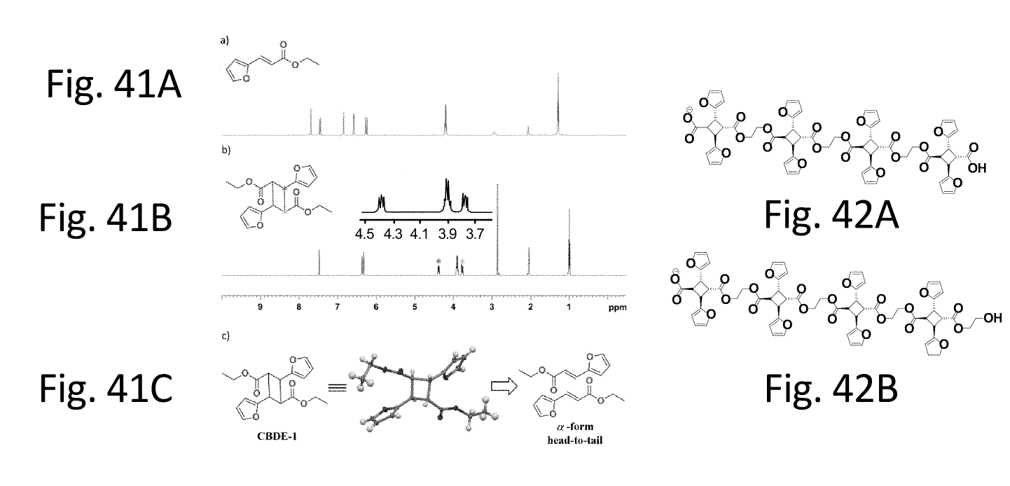

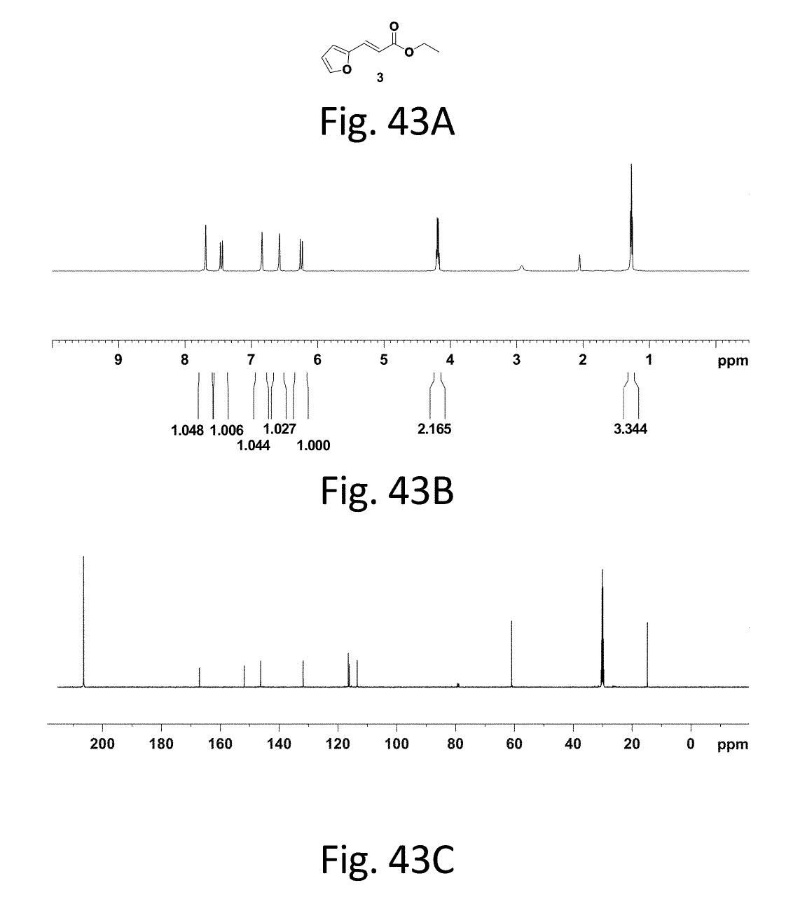

FIGS. 41A-41C show chemical structure and proton NMR of intermediate Compound 3 and CBDE-1, and X-ray diffraction analysis for CBDE-1. FIG. 41A shows Compound 3 in aceton-d₆, an intermediary between furfural and CBDE-1. The proton NMR confirms the chemical structure of Compound 3. FIG. 41B shows CBDE-1 in acetone-d₆. The inset of FIG. 41B shows a magnified range between 3.6 and 4.5 ppm. The proton NMR confirms the structure of CBDE-1. NMR results are discussed in more detail with regards to FIGS. 43-45.

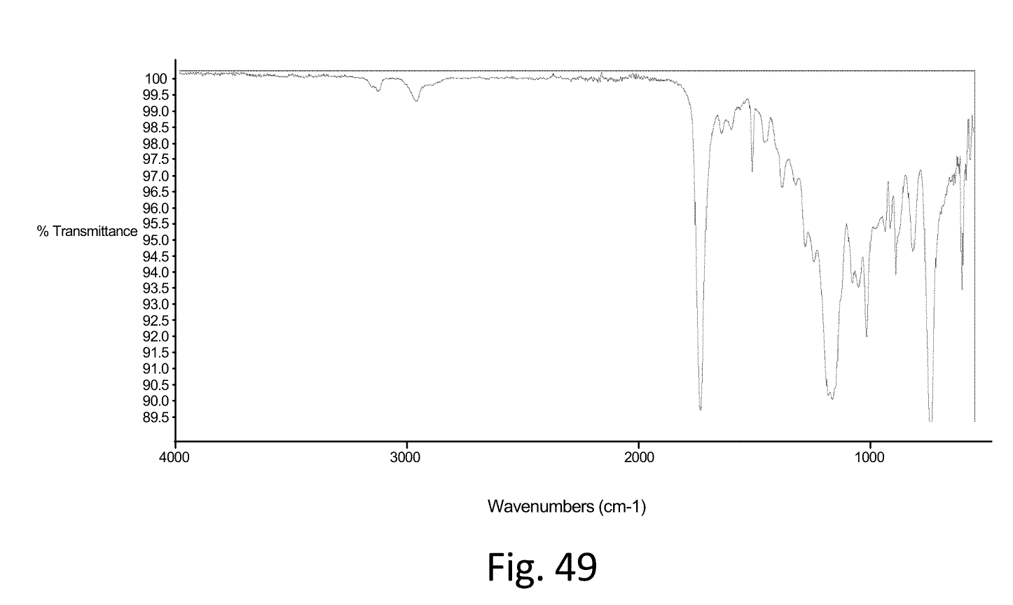

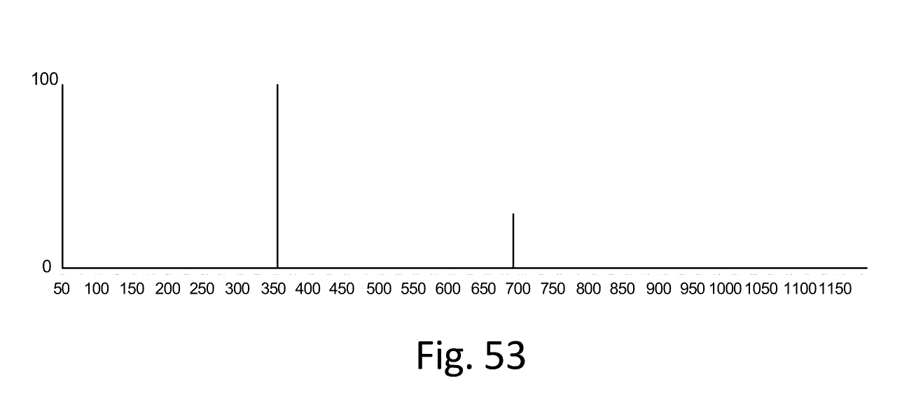

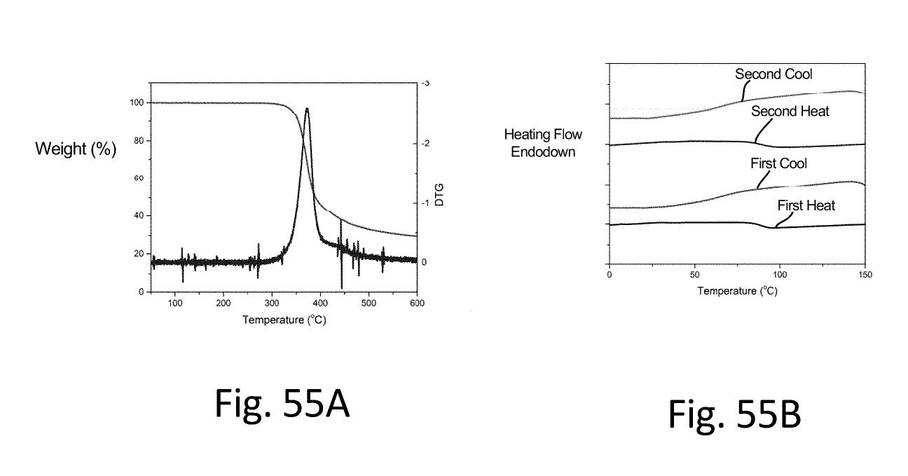

FIGS. 42A-42B show proposed chemical structures of polyethylene cyclobutane-1,3-dicarboxylate (PEC-1), a polyester made from polymerization of CBDA-5. FIG. 42A shows proposed chemical structure of a tetramer of PEC-1 with carboxylic end groups. FIG. 42B shows the anion tetramer of PEC-1 with a hydroxyl end group. These proposed structures are based on FT-IR and MS spectra for PEC-1 after synthesis. The spectra are shown and discussed with reference to FIGS. 49 and 53. Additionally, TGA and DSC data for PEC-1 is shown in FIG. 55A, and DSC data for PEC-1 is shown in FIG. 55B.

FIGS. 43-57 show spectra and data completed in laboratory testing of Intermediate Compound 3, CBDE-1, CBDA-5, and polymer PEC-1. The spectra and data of FIGS. 43-57 confirm the chemical structures proposed in FIGS. 36-42.

FIGS. 43-45 show NMR spectra of Intermediate Compound 3, CBDE-1, and CBDA-5. The peaks in ¹H—, ¹³C—, DEPT-135, DEPT-90, and COSY NMR of these compounds confirms the chemical structures shown in FIGS. 36-41.

FIG. 43A shows the chemical structure of Compound 3, and FIGS. 43B-43C show NMR spectra of Compound 3 which confirm this chemical structure. FIG. 43B shows ¹H-NMR of Compound 3 in acetone-d₆. FIG. 43C shows ¹³C-NMR of Compound 3 in acetone-d₆.

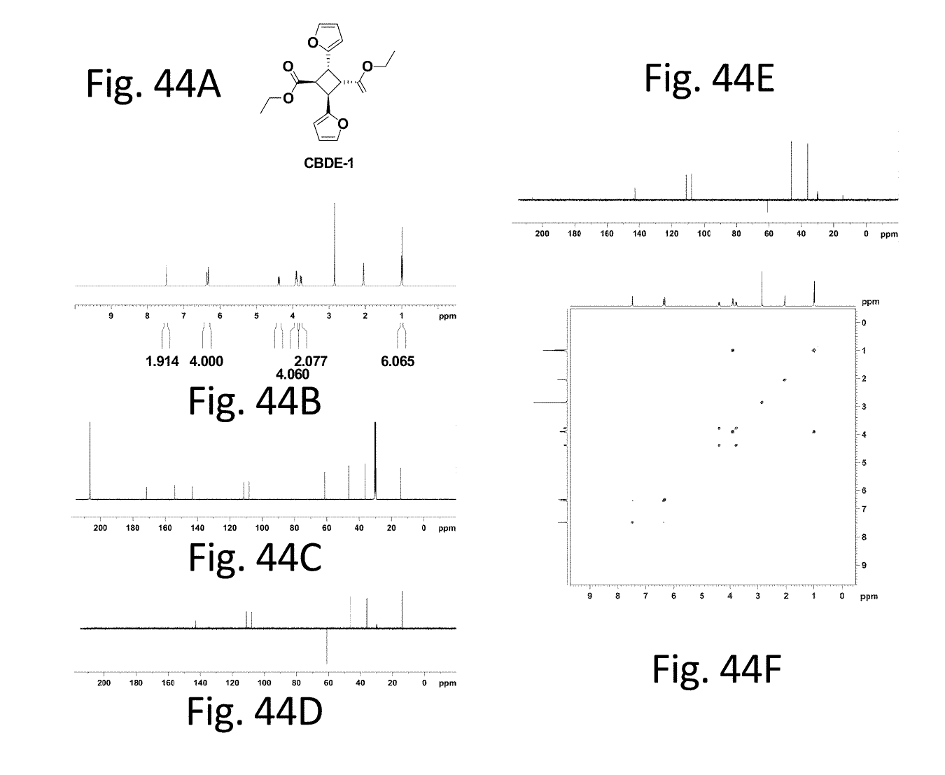

FIG. 44A shows the chemical structure of CBDE-1, and FIGS. 44B-44F show NMR spectra of CBDE-1 which confirm this chemical structure. FIG. 44B shows ¹H-NMR of CBDE-1 in acetone-d₆. FIG. 44C shows ¹³ C-NMR of CBDE-1 in acetone-d₆. FIG. 44D shows DEPT-135 NMR of CBDE-1 in acetone-d₆. FIG. 44E shows DEPT-90 NMR of CBDE-1 in acetone-d₆. FIG. 44F shows COSY NMR of CBDE-1 in acetone-d₆.

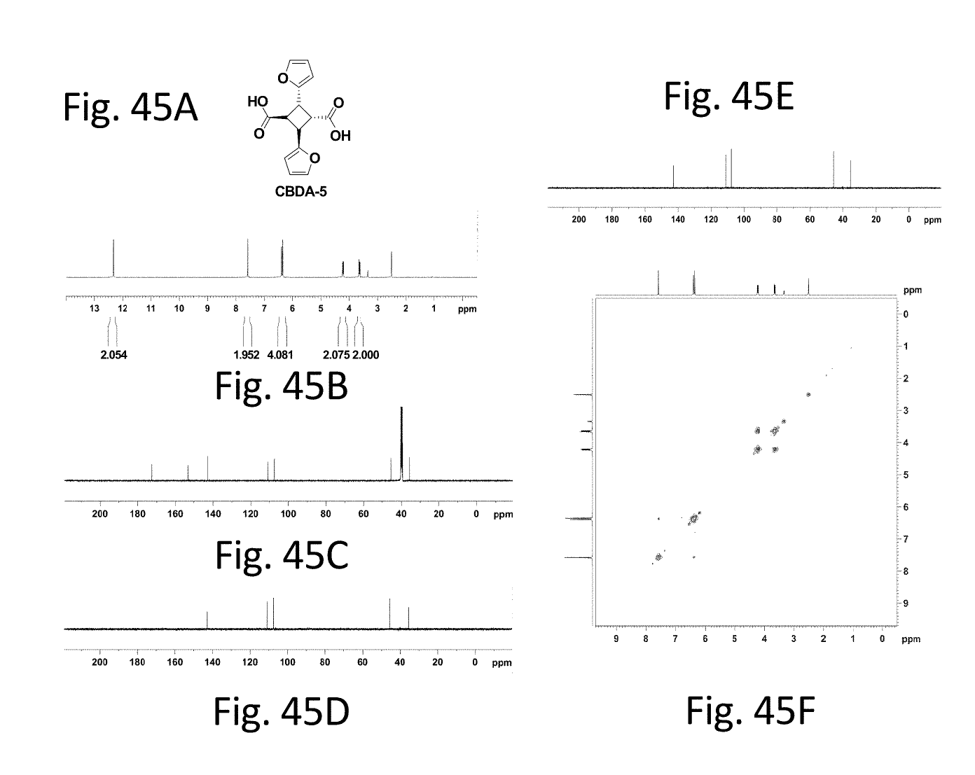

FIG. 45A shows the chemical structure of CBDA-5, and FIGS. 45B-45F show NMR spectra of CBDA-5 which confirm this chemical structure. FIG. 45B shows ¹H-NMR of CBDA-5 in DMSO-d₆. FIG. 45C shows ¹³C-NMR of CBDA-5 in DMSO-d₆. FIG. 45D shows DEPT-135 NMR of CBDA-5 in DMSO-d₆. FIG. 45E shows DEPT-90 NMR of CBDA-5 in DMSO-d₆. FIG. 45F shows COSY NMR of CBDA-5 in DMSO-d₆.

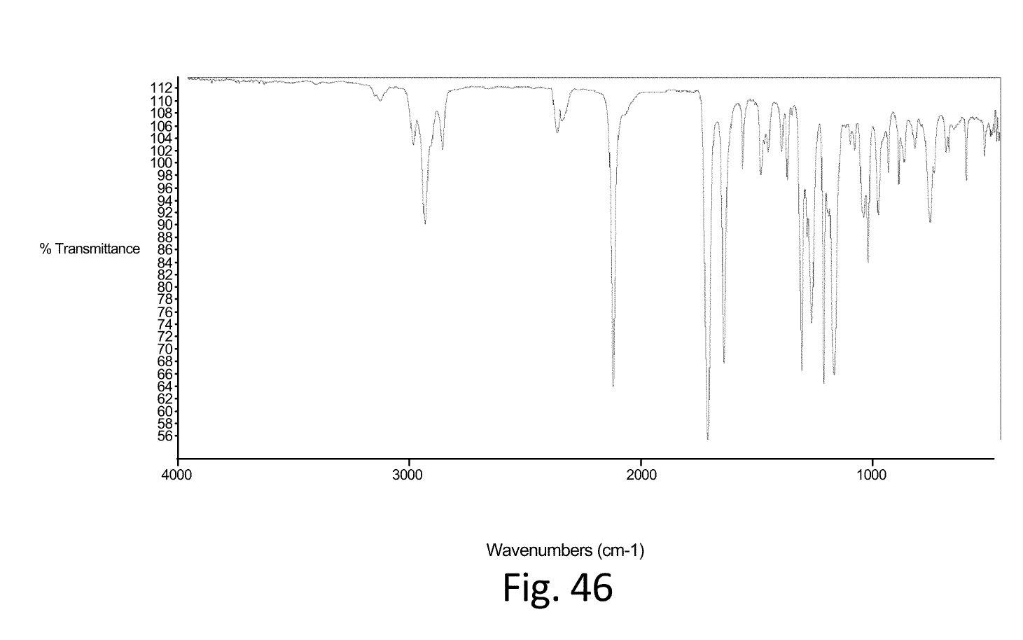

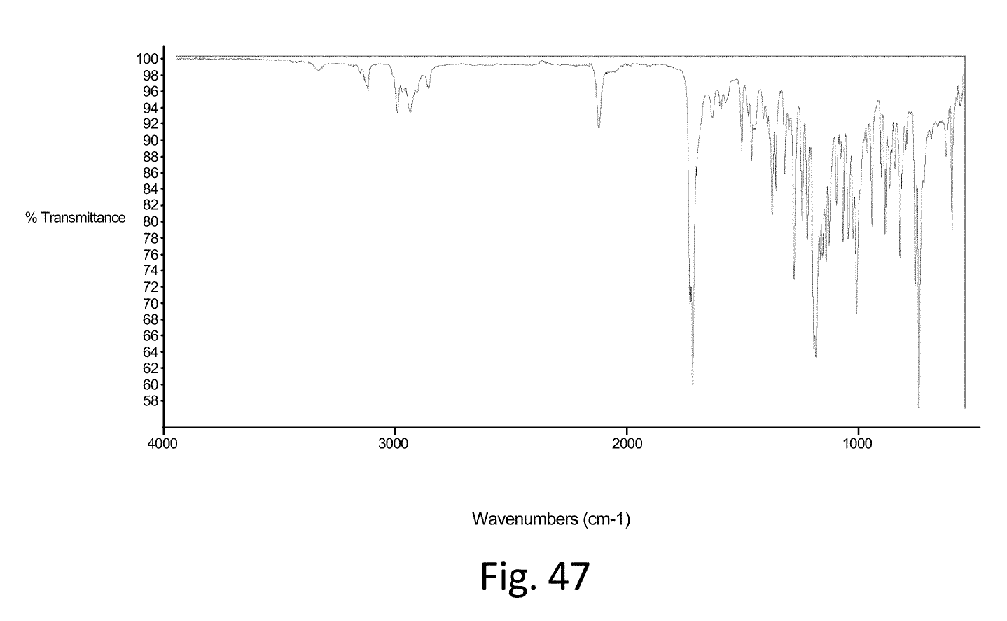

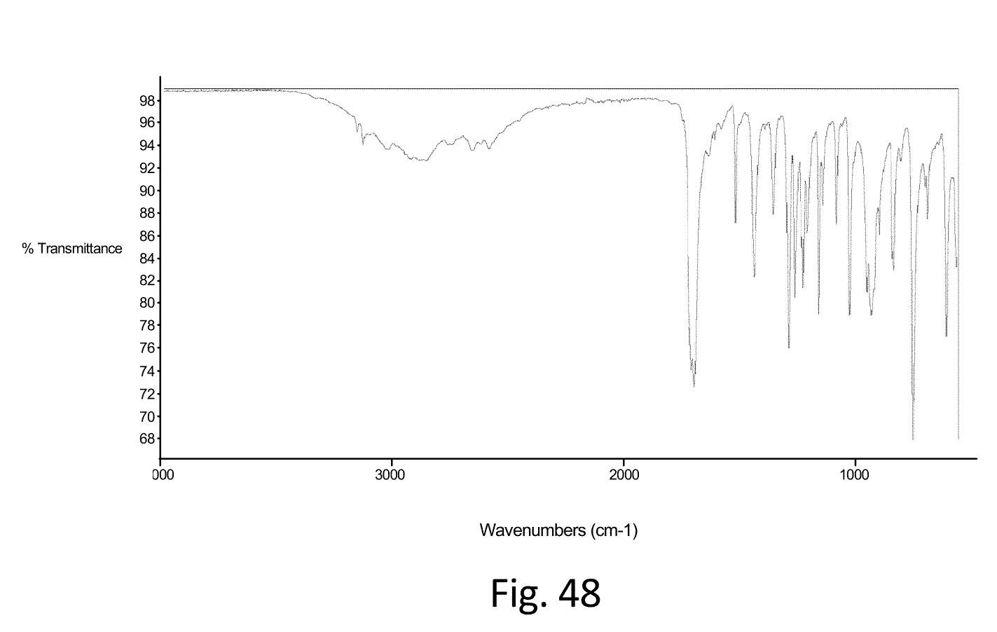

FIGS. 46-49 show FT-IR spectra of Intermediate Compound 3, CBDE-1, CBDA-5, and polymer PEC-1. FIG. 46 shows an FT-IR spectrum for Compound 3. FIG. 47 shows an FT-IR spectrum for CBDE-1. FIG. 48 shows an FT-IR spectrum for CBDA-5. FIG. 49 shows an FT-IR spectrum for PEC-1.

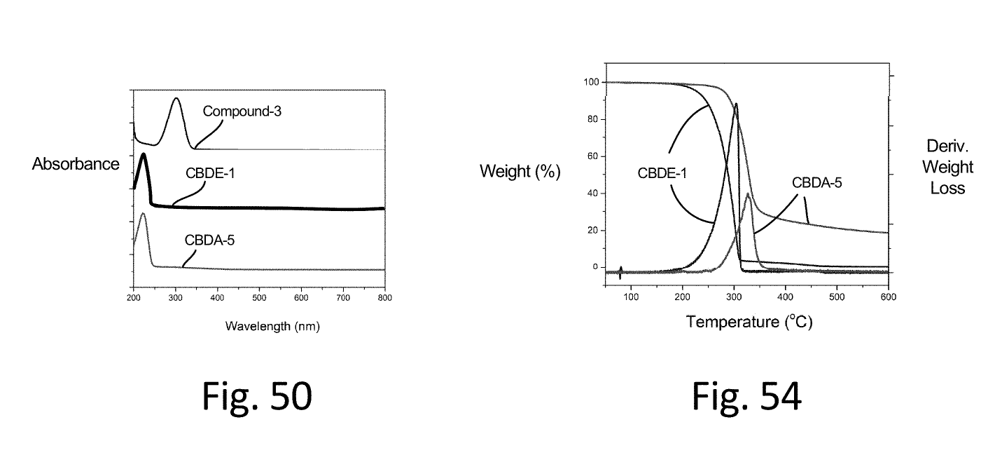

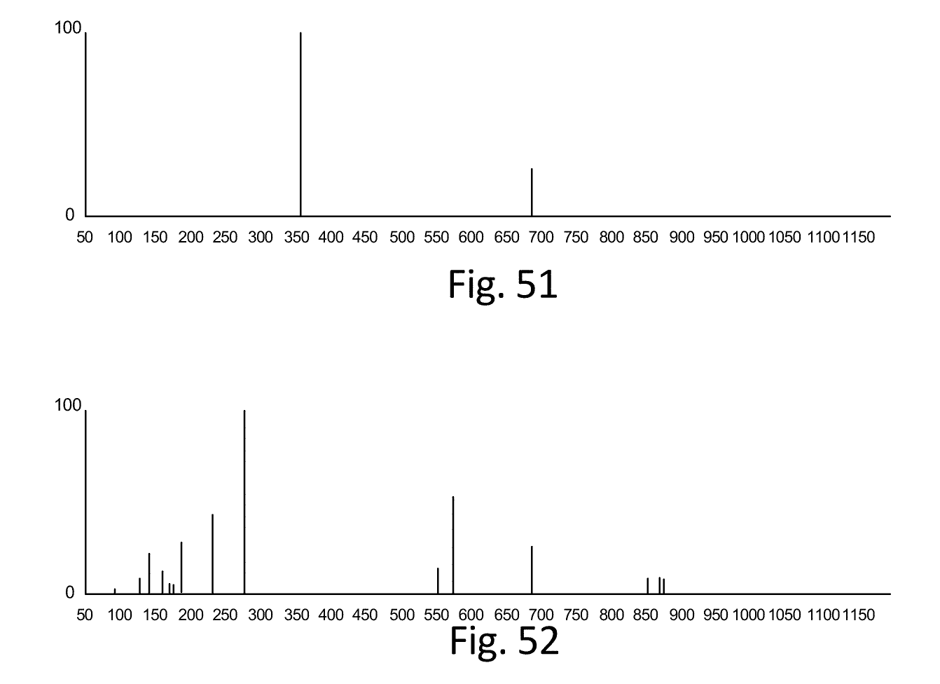

FIG. 50 shows a UV-Vis spectrum of Compound 3, CBDE-1, and CBDA-5.FIGS. 51-53 show MS spectra for CBDE-1, CBDA-5, and PEC-1. FIG. 51 shows an MS spectrum of CBDE-1. FIG. 52 shows an MS spectrum of CBDA-5. FIG. 53 shows an MS spectrum of PEC-1.

FIG. 54 shows TGA and DTG data for CBDE-1 and CBDA-5. FIG. 55A shows TGA and DTG data for PEC-1. FIG. 55B shows DSC data for PEC-1. DSC was recorded from 0° C. to 150° C. with a heating rate of 10° C. per minute under a nitrogen atmosphere.

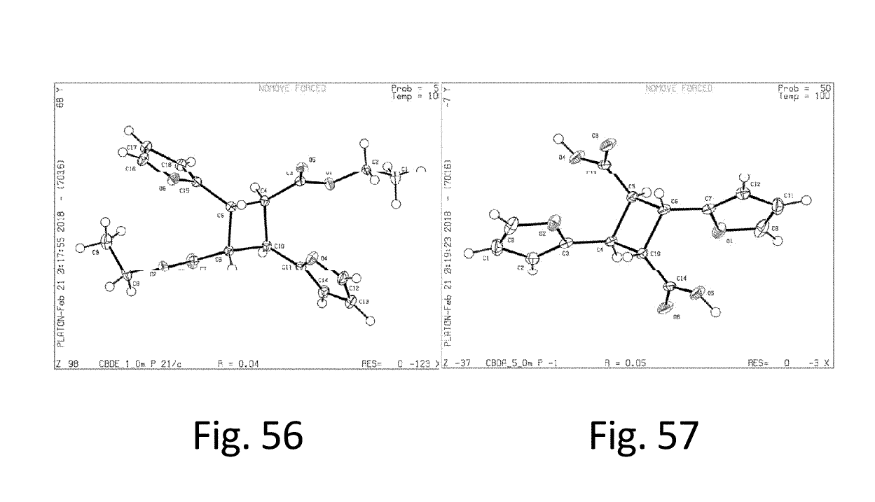

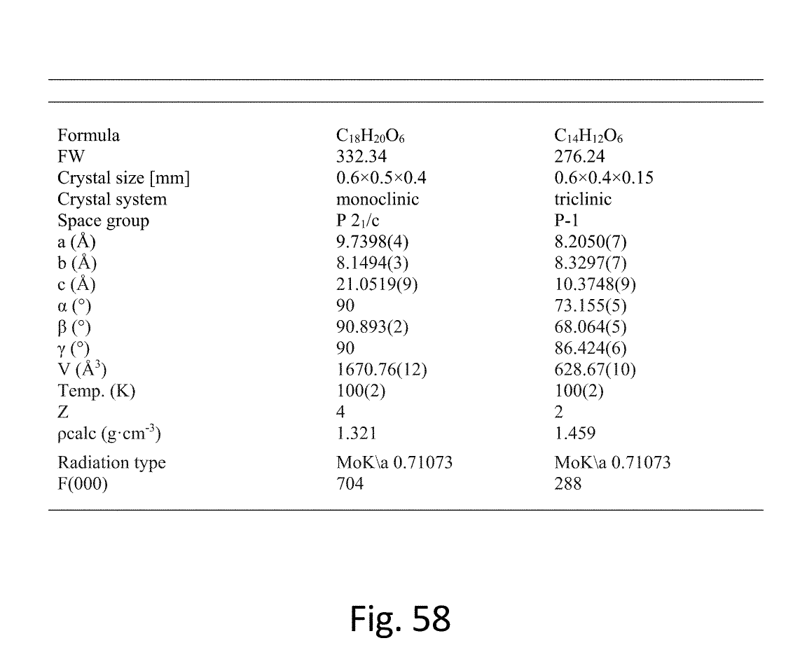

FIG. 56 shows crystal data of CBDE-1. FIG. 57 shows crystal data of CBDA-5. FIG. 58 is a table depicting crystal data of CBDE-1 and CBDA-5, and summarizes many results shown in earlier spectra.

### Synthesis of CBDA-5

Prior to synthesis of polymers with CBDA, a useable CBDA monomer must be synthesized. Furfural obtained from lignincelluslose biomass can be converted into CBDA-5 via photocycloaddition reaction. This process is pictured in FIG. 40.

First, 2-furanacrylic acid is synthesized from furfural. This synthesis starts from Knoevenagel condensation between furfural with malonic acid, which can also be obtained from biomass. The obtained 2-furfranacrylic acid is dimerized through photocycloaddition reaction in the solid state to produce (1R,2S,3R,4S)-rel-3,4-di(furan-2-yl)cyclobutane-1,2-dicarboxylic acid (CBDA-2).

2-furfranacrylic acid is first converted to ethyl 2-furanacrylate. The 2-furfranacrylic acid is mixed with DCC (N,N′-Dicyclohexylcarbodiimide) and DMAP (4-Dimethylaminopyridine) in DCM (Methylene dichloride). Anhydrous ethanol is added to this mixture and stirred at room temperature for 6 hours. The DCM is removed by roller evaporator, then the residue is diluted with water and extracted with hexane. The combined organic layers are washed with brine, dried over sodium sulfate, and filtered through silicone gel pad. The solvent is evaporated to produce ethyl 2-furanacrylate.

To achieve the head-to-tail packing necessary to produce a CBDA molecule, ethyl 2-furanacrylate is prepared in a crystal form using low temperature solvent-free crystallization techniques. This resulting intermediate Compound 3 is used in photocycloaddition. The UV-Vis spectrum of Compound 3 (FIG. 50) shows Compound 3 has an absorbance range between 250 nm and 350 nm. Thus, black light should be used as an ECO-UV source for the photodimerization to synthesize CBDE-1 (the diester of CBDA-5) from Compound 3.

Throughout the photoreaction, FT-IR and ¹H-NMR can be used to confirm the chemical structures of intermediary compounds. The FT-IR (FIGS. 46-49), show that band 1640 cm⁻¹, corresponding to C═C stretching, disappears after photodimerization.

¹H-NMR spectra (FIGS. 43-45) show the double bond peaks 7.43 ppm (d, Ar—CH═) and 6.23 ppm (d, ═CHCOOEt) disappear after photoreaction. In contrast, two new peaks 4.38 ppm (dd, Ar—CH) and 3.77 ppm (dd, —CHCOOEt) arise, which correspond to the sp³ hybridized carbons in the newly formed cyclobutane ring. Consistent results are observed in ¹³C— and DEPT 90 NMR spectra (also in FIGS. 43-45). The appearance of peaks at 46.5 ppm and 36.4 ppm in these spectra are attributable to the formation of the cyclobutane moiety. This means that the cyclobutane ring of the CBDA complex is correctly being formed throughout synthesis.

The deconjugation during the photoreaction from Compound 3 to the dimer CBDE-1 is observed in UV-Vis spectra (FIG. 50) where the λ max of CBDE-1 has a clear blue shift (hypsochromic-shift) compared to that of Compound 3. Specifically, in Compound 3, the furan ring and carbonyl groups are conjugated through the double bond forming a continuous conjugation system before photocycloaddition. The newly formed cycicobutane ring in CBDE-1 cuts off the conjugation chain and results in the blue shift observed from CBDE-1. This confirms the conversion of Compound 3 to CBDE-1. The conversation from Compound 3 to CBDE-1 is approximately 73% in 43 hours and the unreacted Compound 3 can be recovered.

Further analysis during synthesis can be conducted with X-ray diffraction, shown in FIGS. 41 and 56-58. It is difficult to obtain high quality single crystals of Compound 3 for X-ray diffraction due to the low melting point of Compound 3. However, the α form of Compound 3 (that is, head to tail packing of Compound 3, which is desirable for the synthesis of CBDA) is indirectly confirmed via single crystal X-ray diffraction analysis of its dimer, CBDE-1 (FIGS. 41C, 56). The single crystals of CBDE-1 are obtained at the interface of hexane and water solution (FIG. 58). Based on the X-ray diffraction of CBDE-1, the cyclobutane ring of CBDE-1 has a torsion angle of 9° and the four interior angles are 89.1°, 88.9°, 90.0°, and 90.6°. This X-ray crystal structure of CBDE-1 confirms the formation of the head-to-tail dimer of Compound 3 in a solvent-free photoreaction.

Finally, hydrolysis of CBDE-1 yields CBDA-5. CBDE-1 is added to a solution of sodium hydroxide and ethanol, heated to reflux, and cooled to room temperature. The mixture is concentrated to remove ethanol, and the residue is added to water. The aqueous solution is pH adjusted. The precipitate is filtered and washed, resulting in CBDA-5. Analysis of produced CBDA-5, its structure and properties, and why it is a suitable polyester building block, is discussed below in references to FIGS. 38-39, 45, 48, 50, 52, 54, and 57-58.

### Single Crystal Structure X-ray Diffraction Analysis of CBDA-5

X-ray diffraction analysis of produce CBDA-5 monomers allows for confirmation of chemical structure and geometric orientation of the functional groups within CBDA-5. Information about this chemical structure, including the spacing and placement of the two carboxylic acid groups on CBDA-5, indicates that CBDA-5 monomers are suitable for creating strong polymer backbones when polymerized.

For single crystal X-ray diffraction analysis, Single crystals of CBDA-5 are prepared from ethanol solution with one drop of acetic acid. CBDA-5 is a head-to-tail dimer after photodimerization of 2-furanacrylic acid. The chemical structures of CBDA-5 is shown in FIGS. 36, 37, and 40. X-ray single crystal analysis showed the cyclobutane ring in CBDA-5 adopted a near planar conformation with a torsion angle of 1.5° (see FIGS. 39A-39E, 57, and 58).

The cyclobutane ring in CBDA-5 has an exchangeable conformation between coplanar and puckered, where the cyclobutane ring changes shape. This means CBDA-5 has semi-rigid properties, which are desirable for polymer formation. The four interior angles of the cyclobutane ring are 89.2°, 90.8°, 89.2°, and 90.8°. The single crystal X-ray structure of CBDA-5 shows that the two furan groups are in a linear orientation (shown in FIG. 39B).

The interatomic distance between two carboxylic acid groups in CBDA-5 is 4.70 Å, which is shorter than the distance of 4.83 Å in furandicarboxylic acid (FDCA) (pictured in FIG. 36). While the FDCA structure shows an angle of 129.4° between the two carboxylic acid groups, the two carboxylic acid groups in CBDA-5 are pointing towards opposite direction and offset by about 1.31 Å (see FIG. 39B).

The side view of CBDA-5 in FIG. 39D is similar to succinic acid (SA) (shown in FIG. 36). However, the distance of the carboxylic acid groups in CBDA-5 is 0.85 Å longer than in SA. The special orientation of the two carboxylic acid groups in CBDA-5 is suitable for building backbones of linear polymers.

Additionally, a linear supramolecar complex of CBDA-5 can be formed via hydrogen bonding (FIG. 39E). This shows the potential of CBDA-5 crystals for preparing supramolecular materials such as hydrogen-bonded organic frameworks. Overall, the structure or CBDA-5 monomers is suitable for creating polymer backbones because the cyclobutane ring is semi-rigid and the carboxylic acid groups are spatially situated to induce strong bonds.

### Thermostability, chemical and photochemical stability analysis of CBDA-5

The thermal properties and photochemical stability of CBDA-5 and its diester CBDE-1 were also studied. Based on these thermal and photochemical properties, both CBDE-1 and CBDA-5 are suitable renewable building blocks for bio-based materials. FIGS. 38A-38B thermogravimetric analysis (TGA) and differential scanning calorimetry analysis (DSC) of CBDA-5 and CBDE-1.

CBDA-5 and CBDE-1 were studied by TGA from 50 to 600° C. under a nitrogen atmosphere. FIG. 38A shows that CBDE-1 started decomposition at a lower temperature (approximately 183° C.) than CBDA-5 (approximately 202° C.). Both CBDE-1 and CBDA-5 had single stage decomposition, with maximal decomposition rates at approximately 303° C. and 327° C. respectively. Thus, CBDA-5 does not decompose until it reaches higher temperature ranges.

DSC study showed the melting points of CBDE-1 and CBDA-5 are 73° C. and 235° C., respectively. CBDE-1 has a much lower melting point than CBDA-5, which can be attributed to the intermolecular hydrogen bonds between carboxylic acid groups in CBDA-5. CBDE-1 is lacking hydrogen bonds between molecules. The DSC curves of CBDE-1 and CBDA-5 were consistent with their TGA results. The thermostability of both CBDE-1 and CBDA-5 make them suitable for many materials applications.

### Synthesis of CBDA Polymers with CBDA-5

CBDA-5 or CBDE-1 can be used to create a polymer polyethylene cyclobutane-1,3-dicarboxylate (PEC-1) as shown in FIG. 37. Either CBDA-5 or CBDE-1 diols can be used for polymerization in transesterification (of CBDA-5) or polycondensation (of CBDE-1). The produced polyester PEC-1 had good thermal properties and could be used in a variety of industry applications.

Polymerization involves two stages. First, oligomers are created with a catalyst. Second, the solution is processed in a vacuum. In the first stage, synthesis begins with CBDA-5 and ethylene glycol purged with argon. A predetermined solution of titanium isopropoxide in toluene is added to the mixture and then the mixture is heated in a silicon-oil bath to 160° C. for six hours under argon flow to form oligomers.

In the second stage, toluene is collected, and polycondensation is carried out at 200° C. while a vacuum was applied to the reaction mixture for four hours. The viscosity of the reaction mixture keeps increasing in the second stage. After the reaction is completed, the mixture was cooled to room temperature.

Gel permeation chromatography analysis showed the product was a mixture of polymer and oligomer. Thus, a yellowish solid is obtained, and suspended in acetone. A product is precipitated out in water. The produced PEC-1 polymer is produced after drying in a vacuum. FIGS. 42A-42B depict proposed chemical structures of produced PEC-1.

Like the production of polymers from CBDA-1, the production of PEC-1 from CBDA-5 requires a linker molecule containing both a non-reactive R group and at least two reactive X groups. The X groups of the linker molecule react with the carboxylic acids on the CBDA-5 molecules, and polymerize the CBDA-5 molecules in PEC-1. In the described methodology above, the linker molecule was in the solution of titanium isopropoxide in toluene. However, a variety of linker molecules can be used to produce varying types of polymers and polyesters from CBDA-5. These variants are discussed in more depth below under “Modification of CBDA Polymers.”

### Discussion of PEC-1 Results

PEC-1 is a thermally and chemically stable polymer produced from CBDA-5 building blocks, originally derived from lignocellulose biomass. PEC-1 polyester made from CBDA-5 (or CBDE-1) building blocks through transesterification (or polycondensation) was analyzed with TGA, DTG, DSC (FIGS. 55A-55B), FT-IR (FIG. 49), and MS (FIG. 53) to verify the thermal and chemical stability of PEC-1

Thermostability analysis of PEC-1 with TGA, DTG, and DSC showed the polymer was thermally stable. PEC-1 had 50% weight retention at 400° C., 5% and 10% weight loss were at 345° C. and 355° C., respectively. The corresponding DTG curves of PEC-1 show the maximum decomposition occurred at 375° C. The char yield of PEC-1 at 600° C. was about 30%.

DSC of PEC-1 showed the glass transition temperature was 88° C., which is similar to other polymers such as polyethylene 2,5-furandicarboxylate (PEF) (at 85° C.) but higher than polyethylene terephthalate (PET) (at 76° C.). In contrast, glass transition temperatures of polybutylene succinate (PBS) is about −30° C. due to the high flexibility of the aliphatic polyester chain. Both TGA and DSC results indicate that PEC-1 is thermally stable.

The chemical structure analysis was done through Mass Spectra (MS). In positive MS mode, the MS of PEC-1 is very similar to that of CBDA-5. The negative MS for PEC-1 (FIG. 53) is more useful for analysis due to the two carboxylic acid groups in CBDA-5.

The negative ion MS (FIG. 53) shows two series peaks, and both have a repeating m/z 302 which is consistent with the unit mass of PEC-1 (C₁₆H₁₄O₆, 302.0790). One of the series peaks is 302.0790×n+275, which suggests that a linear polyester was obtained with a carboxylic acid group as the end group. The chemical structure for this polyester is shown in FIG. 42A. A peak with 1181.2927 is found where n is 3 (m/z=302.0790×3+275). The proposed corresponding structure is pictured in FIG. 42A with two carboxylic acid end groups where one of the groups is an anion. Another series of peaks is 302.0790×n+17, which indicates the end group of the polymer is a hydroxyl. This proposed structure is pictures in FIG. 42B.

The monoisotopic mass of the proposed chemical structure for PEC-1 is 1225.3194, and the found mass is 1224.3191 in the MS, which suggests a tetramer of PEC-1 is produced. These MS results show CBDA-5 can be successfully used in linear polyester preparation, such as in creation of PEC-1.

The use of CBDA-5 turns biomass into valuable chemical building blocks for polymer production. The use of furfural from lignocellulosic biomass (such as saw dust, corncobs, or wheat bran) to produce CBDA allows for its use in materials applications. The CBDA-5 building block has a semi-rigid structure that produces fully bio-based polyesters.

### III. Modification of Cbda Polymers

CBDA polymers (poly-α-truxillate or PEC-1) are unique compounds with a variety of industry applications where polymers or polyesters are typically used, including materials synthesis. The required starting materials for CBDA polymers are readily available, and can include biomass. Polymers produced from both CBDA-1 and CBDA-5 can be made with a variety of linker molecules. Additionally, the functional groups on the CBDA building blocks can be tailored to specific polymer needs.

In synthesis of CBDA polymers, a variety of non-reactive R groups and reactive X-groups can be used to link together CBDA monomers as shown in FIG. 2. Non-reactive R groups, residing in the center of the linker molecule, can includes a variety of chemical groups. R groups can be aliphatic chains, such as methylene, ethylene, propylene, butylene, pentylene, hexylene, heptylene, octylene with a general structure of —(CH₂)ₙ—, where n is an integer. Exemplary R groups for use in the material synthesis also include aliphatic heterochains (such as —CH₂ CH₂OCH₂CH₂—, —CH₂ CH₂OCH₂CH₂OCH₂CH₂—, —CH₂CH₂SCH₂CH₂—, CH₂CH₂NHCH₂CH₂—), branched aliphatic chains or heterochains (such as —CH(CH₃)CH₂—, —CH(CH₃)CH₂CH₂—, —CH₂CH(CH₃)CH₂—, —CH(CH₂CH₂CH₃)CH₂—, —CH(CH₃)CH₂C(CH₃)₂—, —CH₂CH(OH)CH₂—, —CH₂CH(NH₂)CH₂—), linking molecules containing substituted and unsubstituted aliphatic rings (such as —C₆H₁₀—, —CH₂(C₄H₆)CH₂—, —CH(C(CH₃)₂)₂CH— (or 2,2,4,4-tetramethyl-1,3-cyclobutanyl)), linking molecules containing substituted or unsubstituted aromatic ring (such as —C₆H₄—, —CH₂(C₆H₄)CH₂—, —CH₂(C₄H₂O)CH₂— (or furan-2,5-dimethylene), —CH₂(C₄H₂S)CH₂— (or thiophene-2,5-dimethylene), —CH₂(C₄H₂NH)CH₂— (or pyrrole-2,5-dimethylene)), substituted or unsubstituted heterocyclic rings (such as dioxane, tetrahydrofuran, pyridine, furan, or thiophene), and combinations of any of the above (such as—CH₂C₆H₄CH₂OCH₂CH₂).

Each linker molecule can include at least two X groups (see FIG. 2) that will reach with the CBDA diacid groups to polymerize. The X groups can be all the same, all different, or a combination thereof. Suitable X groups include nitrogen-containing functional groups (including, but not limited to, amine, imine, oxime, imide, cyanates, isocyanate, azide, azo), oxygen-containing functional groups (including, but not limit to, hydroxy, carboxyl, carboxylate, and epoxide-containing groups), halogens, carbon containing functional groups (including, but not limit to, alkyne), sulfur-containing functional groups (including, but not limit to, thiol or thiocyanate), boron-containing functional groups, phosphorus-containing functional groups, metals and metal cations (including, but not limit to, zinc, copper, calcium, magnesium, aluminum, iron, manganese, nickel, lithium, sodium, or potassium ions), and combinations of any of above (such as chloropyridinyl, vanillyl, —CHBrCH₂N═C═O, or —CHN₂—, isosorbide, starch, glucose, cyclodextrin, or amino acid).

The selection of specific R and X groups in linker molecules used to polymerize CBDA building blocks offers the ability to tailor the type of polymer being built. Different R and X groups allow for variants in polymer thermal, chemical, and structural properties. This ultimately results in different industry applications.

Additionally, the functional groups FG on CBDA molecules can vary (see FIG. 2). Not including the diacid groups on CBDA molecules, the cyclobutane ring of CBDA can host up to six different functional groups FG. The functional groups FG can be all the same, all different, or any combination thereof

Exemplary functional groups FG includes groups used for materials synthesis such as hydrogen, halogen, and unsubstituted or substituted hydrocarbyl groups such as —CH₃, —CH₂CH₃, —CH(CH₃)₂, —C₆H₁₁, —CH═CHCH₃, —CH₂OH, or —CH₂NH₂. Alternatively, functional groups FG can include -phenyl, -furyl, -thiopenyl, or -pyridinyl groups; nitrogen, oxygen, sulfur, boron, silicon, or phosphorous-containing groups (such as —NO₂, —OH, OCH₃, —NH₂, N(CH₃), —Si(CH₃)₃, —B(CH₃)₂); or any combination of the above groups (such as —C₇H₇O₂((4′-hydroxy-3′-methoxy-)phenyl), —CH₂N(CH₃)₂ 5-nitrofuran-2-yl, —OSi(CH₃)₃, and —B(OCH₃)₂). Additionally, on the CBDA molecules, the dicarboxylic acid can be converted to its derivatives, such as ester, acid chloride, and anhydride for materials preparation.

Overall, the use of CBDA semi-rigid building blocks produces a variety of tailorable, thermally and chemically stable polymers. Both CBDA-1 and CBDA-5 can be produced from readily available materials, with the added benefit that production of CBDA-5 turns biomass into valuable chemical feedstock.

### Discussion of Possible Embodiments

The following are non-exclusive descriptions of possible embodiments of the present invention.

A method of making a polyester includes providing a plurality of cyclobutane-1,3-diacid molecules, and polymerizing the plurality of cyclobutane-1,3-diacid molecules together with a plurality of linker molecules to create a polymer. Each of the plurality of linker molecules includes an R group and at least two X groups. The R group does not react with the cyclobutane-1,3-diacid molecules. The X groups do react with the cyclobbutane-1,3-diacid molecules.

The method of the preceding paragraph can optionally include, additionally and/or alternatively, any one or more of the following features, configurations and/or additional components:

The X groups reacts with a carboxylic acid of the cyclobutane-1,3-diacid molecule.

Each of the plurality of linker molecules comprises at least two X groups.

The X groups are selected from the group consisting of nitrogen containing functional groups, oxygen containing functional groups, halogens, sulfur containing functional groups, boron containing functional groups, phosphorus containing functional groups, metals, metal cations, and combinations thereof

The R group is selected from the group consisting of an aliphatic chain, an aliphatic heterochain, a branched aliphatic chain, an aliphatic ring, an aromatic ring, a heterocyclic ring, and combinations thereof

Polymerizing the plurality of cyclobutane-1,3-diacid molecule together comprises: forming a series of alcohols through condensation reactions, each of the series of alcohols comprising at least two hydroxy groups.

The plurality of cyclobutane-1,3-diacid molecules are (1α,2α,3β,4β)-2,4-diphenylcyclobutane-1,3-dicarboxylic acid.

The plurality of cyclobutane-1,3-diacid molecules are (1α, 2α, 3β, 4β)-2,4 -di(furan-2-yl)cyclobutane-1,3-dicarboxylic acid.

The plurality of cyclobutane-1,3-diacid molecules are cyclobutane-1,3-diester.

The plurality of cyclobutane-1,3-diacid molecules each further comprise one or more functional groups selected from the group consisting of hydrogen, halogens, hydrocarbyl, phenyl, furyl, thiophenyl, pyridinyl, nitrogen containing groups, oxygen containing groups, sulfur containing groups, boron containing groups, silicon containing groups, phosphorus containing groups, and combinations thereof.

The method includes synthesizing the plurality of cyclobutane-1,3-diacid molecules.

Synthesizing the plurality of cyclobutane-1,3-diacid molecules comprises photodimerization in the solid-state from trans-cinnamic acid.

Synthesizing the plurality of cyclobutane-1,3-diacid molecules comprises photodimerization in the solid state from furfural.

A polymer includes a plurality of cyclobutane-1,3-diacid monomers, and a plurality of linking groups. Each of the plurality of cyclobutane-1,3-diacid monomers contains two carboxylic acid groups. Each of the plurality of linking groups includes an R group and at least two X groups. Each of the X groups is connected to one of the carboxylic acid groups in the plurality of cyclobutane-1,3-diacid monomers such that each of the plurality of linking groups chemically bonds to at least two of the plurality of cyclobutane-1,3-diacid monomers.

The polymer of the preceding paragraph can optionally include, additionally and/or alternatively, any one or more of the following features, configurations and/or additional components:

The X groups are selected from the group consisting of nitrogen containing functional groups, oxygen containing functional groups, halogens, carbon containing functional groups, sulfur containing functional groups, boron containing functional groups, phosphorus containing functional groups, metals, metal cations, and combinations thereof

The R group is selected from the group consisting of an aliphatic chain, an aliphatic heterochain, a branched aliphatic chain, an aliphatic ring, an aromatic ring, a heterocyclic ring, and combinations thereof

The cyclobutane-1,3-diacid monomers are derived from (1α,2α,3β,4β)-2,4 -diphenylcyclobutane-1,3-dicarboxylic acid, (1α,2α,3β,4β)-2,4-di(furan-2-yl)cyclobutane-1,3 -dicarboxylic acid, or cyclobutane-1,3-diester.

The cyclobutane-1,3-diacid monomers further comprise one or more functional groups selected from the group consisting of hydrogen, halogens, hydrocarbyl, phenyl, furyl, thiophenyl, pyridinyl, nitrogen containing groups, oxygen containing groups, sulfur containing groups, boron containing groups, silicon containing groups, phosphorus containing groups, and combinations thereof.

The dicarboxylic acid groups of the cyclobutane-1,3-diacid monomers are converted to a derivative.

The derivative is an ester, an acid chloride, or an anhydride.

While the invention has been described with reference to an exemplary embodiment(s), it will be understood by those skilled in the art that various changes may be made and equivalents may be substituted for elements thereof without departing from the scope of the invention In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A method of making a polyester comprises:
providing a plurality of cyclobutane-1,3-diacid molecules; and
polymerizing the plurality of cyclobutane-1,3-diacid molecules together with a plurality of linker molecules to create a polymer, each of the plurality of linker molecules comprising an R group and at least two X groups, wherein the R group does not react with the cyclobutane-1,3-diacid molecules, and wherein the X groups do react with the cyclobbutane-1,3-diacid molecules.

2. The method of claim 1, wherein the X groups react with a carboxylic acid of the cyclobutane-1,3-diacid molecule.

3. The method of claim 1, wherein each of the plurality of linker molecules comprises at least two X groups.

4. The method of claim 1, wherein the X groups are selected from the group consisting of nitrogen containing functional groups, oxygen containing functional groups, halogens, carbon containing functional groups, sulfur containing functional groups, boron containing functional groups, phosphorus containing functional groups, metals, metal cations, and combinations thereof

5. The method of claim 1, wherein the R group is selected from the group consisting of an aliphatic chain, an aliphatic heterochain, a branched aliphatic chain, an aliphatic ring, an aromatic ring, a heterocyclic ring, and combinations thereof

6. The method of claim 1, wherein polymerizing the plurality of cyclobutane-1,3-diacid molecule together comprises: forming a series of alcohols through condensation reactions, each of the series of alcohols comprising at least two hydroxy groups.

7. The method of claim 1, wherein the plurality of cyclobutane-1,3-diacid molecules are (1α,2α,3β,4β)-2,4-diphenylcyclobutane-1,3-dicarboxylic acid.

8. The method of claim 1, wherein the plurality of cyclobutane-1,3-diacid molecules are (1α,2α,3β,4β)-2,4-di(furan-2-yl)cyclobutane-1,3-dicarboxylic acid.

9. The method of claim 1, wherein the plurality of cyclobutane-1,3-diacid molecules are cyclobutane-1,3-diester.

10. The method of claim 1, wherein the plurality of cyclobutane-1,3-diacid molecules each further comprise one or more functional groups selected from the group consisting of hydrogen, halogens, hydrocarbyl, phenyl, furyl, thiophenyl, pyridinyl, nitrogen containing groups, oxygen containing groups, sulfur containing groups, boron containing groups, silicon containing groups, phosphorus containing groups, and combinations thereof

11. The method of claim 1, further comprising synthesizing the plurality of cyclobutane-1,3-diacid molecules.

12. The method of claim 11, wherein synthesizing the plurality of cyclobutane-1,3-diacid molecules comprises photodimerization in the solid-state from trans-cinnamic acid.

13. The method of claim 11, wherein synthesizing the plurality of cyclobutane-1,3-diacid molecules comprises photodimerization in the solid state from furfural.

14. A polymer comprising:
a plurality of cyclobutane-1,3-diacid monomers, each of the plurality of cyclobutane-1,3-diacid monomers comprising two carboxylic acid groups; and
a plurality of linking groups, each of the plurality of linking groups comprising an R group and at least two X groups, each of the X groups connected to one of the carboxylic acid groups in the plurality of cyclobutane-1,3-diacid monomers such that each of the plurality of linking groups chemically bonds to at least two of the plurality of cyclobutane-1,3-diacid monomers.

15. The polymer of claim 14, wherein the X groups are selected from the group consisting of nitrogen containing functional groups, oxygen containing functional groups, halogens, carbon containing functional groups, sulfur containing functional groups, boron containing functional groups, phosphorus containing functional groups, metals, metal cations, and combinations thereof

16. The polymer of claim 14, wherein the R group is selected from the group consisting of an aliphatic chain, an aliphatic heterochain, a branched aliphatic chain, an aliphatic ring, an aromatic ring, a heterocyclic ring, and combinations thereof

17. The polymer of claim 14 wherein the cyclobutane-1,3-diacid monomers are derived from (1α,2α,3β,4β)-2,4-diphenylcyclobutane-1,3-dicarboxylic acid, (1α,2α,3β,4β)-2,4-di(furan-2-yl)cyclobutane-1,3-dicarboxylic acid, or cyclobutane-1,3-diester

18. The polymer of claim 14 wherein the cyclobutane-1,3-diacid monomers further comprise one or more functional groups selected from the group consisting of hydrogen, halogens, hydrocarbyl, phenyl, furyl, thiophenyl, pyridinyl, nitrogen containing groups, oxygen containing groups, sulfur containing groups, boron containing groups, silicon containing groups, phosphorus containing groups, and combinations thereof

19. The polymer of claim 14 wherein the dicarboxylic acid groups of the cyclobutane-1,3-diacid monomers are converted to a derivative.

20. The polymer of claim 19, wherein the derivative is an ester, an acid chloride, or an anhydride.
